SEQUENCE LISTING (6 sequences)

SEQ ID NO: 1 - protein, 8 aa, Artificial sequence
  Motif sequence
GPLGIAGQ

SEQ ID NO: 2 - protein, 6 aa, Artificial sequence
  Motif sequence
XXXXXX

SEQ ID NO: 3 - protein, 6 aa, Artificial sequence
  Motif sequence
XXXXXX

SEQ ID NO: 4 - DNA, 21 bp, Artificial sequence
  siRNA sequence
aagugguagaagccguagctt

SEQ ID NO: 5 - DNA, 21 bp, Artificial sequence
  siRNA sequence
ccguatcguaagcagtacttt

SEQ ID NO: 6 - DNA, 21 bp, Artificial sequence
  siRNA sequence
agcgcaaccggacgaaucctt

(19) United States
(12) Patent Application Publication
(10) Pub. No.: US 2016/0312218 A1
(43) Pub. Date: Oct. 27, 2016

(54) SYSTEM FOR CO-DELIVERY OF POLYNUCLEOTIDES AND DRUGS INTO PROTEASE-EXPRESSING CELLS

(71) Applicant: NORTHEASTERN UNIVERSITY, Boston, MA (US)

(72) Inventors: Lin ZHU, Kingsville, TX (US); Vladimir Torchilin, Charlestown, MA (US)

(21) Appl. No.: 15/033,996

(22) PCT Filed: Oct. 21, 2014
(86) PCT No.: PCT/US14/61612

§ 371 Date: May 3, 2016

Related U.S. Application Data
(60) Provisional application No. 61/899,511, filed on Nov. 4, 2013.

(51) Int. Cl. C12N 15/113; A61K 45/06; A61K 31/337; A61K 47/48; A61K 9/107; A61K 31/713

(52) U.S. Cl. CPC C12N 15/113; C12N 15/1137; A61K 9/1075; A61K 31/713; A61K 31/337; A61K 47/48215; A61K 47/48338; A61K 47/48192; A61K 45/06; C12N 2320/32; C12N 2310/14; C12N 2310/11; C12N 2320/31; C12N 2310/351; C12N 2330/30; C12N 2310/3513

(57) ABSTRACT

Nanoparticle compositions and pharmaceutical compositions for the delivery of a polynucleotide and a hydrophobic pharmaceutical agent to a cell or tissue that overexpresses a protease are provided. Methods of making such compositions and methods of using such composition to treat a condition associated with a cell or tissue that overexpresses a protease are provided as well. Also provided are kits for use in treating a condition associated with a cell or tissue that overexpresses a protease. The compositions, methods, and kits can be used to selectively deliver anti-tumor agents to cancer cells.

### CROSS REFERENCE TO RELATED APPLICATIONS

This application claims the benefit of U.S. Provisional Application No. 61/899,511, filed Nov. 4, 2013 and entitled “System for Delivery of siRNA and Co-Delivery of siRNA and Drug into MMP-Expressing Cells”, which is hereby incorporated by reference in its entirety.

### STATEMENT REGARDING FEDERALLY SPONSORED RESEARCH OR DEVELOPMENT

This invention was developed with financial support from Grant No. 1R01CA121838 and Grant No. U54CA151881 from the National Institutes of Health and from Grant No. PF-13-361-01-CDD from the American Cancer Society. The U.S Government has certain rights in the invention.

### BACKGROUND

Small interfering RNA (siRNA) has shown therapeutic potential against numerous diseases, including cancer [1-3]. However, the efficiency of siRNA is significantly compromised by its poor stability, short circulation time, non-specific tissue distribution, and insufficient cellular transport [4]. Polyethylenimine (PEI), a cationic polymer, has been widely used in gene and siRNA delivery, due to its excellent transfection capability [5]. To improve the efficiency of siRNA delivery, a lipid-polymer, PEI (1800 Da)-1,2-dioleoyl-sn-glycero-3-phosphoethanolamine (DOPE) (PEI-PE), which possesses the advantages of both PEI and DOPE, has been synthesized [6,7]. However, the high charge of PEI causes the non-selective electrostatic interaction between the nanocarriers and biological molecules or membranes, leading to low tumor targeting. Paclitaxel (PTX), on the other hand, is one of the most commonly used antineoplastic agents. However, its applications are complicated by its low solubility, off-target toxicity and acquired drug resistance. Although various drug delivery systems have been developed, co-delivery of siRNA and hydrophobic drugs like PTX remains a challenge. Usually, because of their distinct physicochemical properties, siRNA and hydrophobic drugs are loaded into individual carriers for simultaneous administration. Since these molecules may not be delivered to the same cell, low synergistic effects are possible [8,9]. To achieve a better synergistic effect, co-delivery of these molecules by the same carrier has been investigated [8-10]. However, the targeted co-delivery of siRNA and drug to tumor cells by the same nanocarrier is rare.

Matrix metalloproteinases (MMPs), especially MMP2, are known to be involved in cancer invasion, progression, and metastasis. The up-regulated MMP2 is considered as a biomarker for diagnostics and prognostics in many cancers, and also has been considered for targeting drug delivery via an enzyme-triggered mechanism [11]. In previous studies, a synthetic octapeptide (GPLGIAGQ) has been used as a stimulus-sensitive linker in both liposomal [12] and micellar nanocarriers [13] for MMP2-triggered tumor targeting.

### SUMMARY OF THE INVENTION

Described herein are molecular compositions, nanoparticle compositions, and pharmaceutical compositions for the delivery of a polynucleotide and a hydrophobic pharmaceutical agent to a cell or tissue that overexpresses a protease. Methods of making such compositions and methods of using such composition to treat a condition associated with a cell or tissue that overexpresses a protease are provided as well. Also described are kits for use in treating a condition associated with a cell or tissue that overexpresses a protease.

In one aspect, the invention is a protease-sensitive, polynucleotide-binding molecule including: an uncharged hydrophilic polymer; a peptide having a target cleavage site for a protease, wherein the peptide is attached to the to the uncharged hydrophilic polymer by a first covalent linkage; a positively-charged polymer, wherein the positively-charged polymer is attached to the peptide by a second covalent linkage, and wherein the positively-charged polymer binds one or more polynucleotide molecules; and a phospholipid, wherein the phospholipid is attached to the positively-charged polymer by a third covalent linkage; wherein the uncharged hydrophilic polymer, the peptide, the positively-charged polymer, and the phospholipid are present in the molecule in about a 1:1:1:1 molar ratio.

In some embodiments, the uncharged polymer may be polyethylene glycol, polyvinylpyrrolidone, or polyacrylamide In an embodiment, the uncharged polymer is polyethylene glycol. In an embodiment, the polyethylene glycol has an average molecular weight from about 1000 to about 5000 daltons. In an embodiment, the polyethylene glycol has an average molecular weight of about 2000 daltons.

In some embodiments, the peptide has a target cleavage site is specific for a matrix metalloproteinase. In some embodiments, the matrix metalloproteinase is MMP-2 or MMP-9. In some embodiments, the peptide comprises the amino acid sequence Gly-Pro-Leu-Gly-Ile-Ala-Gly-Gln (SEQ ID NO:1). In some embodiments, the peptide has a matrix metalloproteinase target cleavage site found in one or more of Aggrecan, Big endothelin-1, Brevican/BEHAB, Collagen-α1(I), Collagen-α1(X), Decorin, FGFR-1, Galectin-3, IGFBP-3, IL-1β, Laminin-5 γ2-chain, α2-Macroglobulin, MCP-3, Pregnancy zone protein, Pro-MMP-1, Pro-MMP-2, SPARC, Substance P, Betaglycan, Dentin, Integrin-αV, Integrin-α6, Integrin-αX, Integrin-α9, NG2 proteoglycan, Neurocan, and PAI-3. In some embodiments, the peptide comprises the sequence Xaa₁-Xaa₂-Xaa₃-Xaa₄-Xaa₅-Xaa₆, wherein Xaa₁ is Ala, Ile, Pro, or Val; Xaa₂ is any amino acid; Xaa₃ is Ala, Asn, Gln, Glu, Gly Ser, or Thr; Xaa₄ is Arg, Ile, Leu, Met, Phe, or Tyr; Xaa₅ is any amino acid; and Xaa₆ is Ala, Gln, Gly, Met, Ser, Tyr, or Val; and wherein the protease cleaves the peptide bond between Xaa₃ and Xaa₄ (SEQ ID NO: 2). In some embodiments, the peptide comprises the sequence Xaa₁-Xaa₂-Xaa₃-Xaa₄-Xaa₅-Xaa₆, wherein Xaa₁ is Ala, Ile, Pro, or Val; Xaa₂ is Ala, Arg, Asn, Glu, Gly, Leu, Met, Phe, Tyr, or Val; Xaa₃ is Ala, Asn, Gln, Glu, Gly Ser, or Thr; Xaa₄ is Arg, Ile, Leu, Met, Phe, or Tyr; Xaa₅ is Ala, Arg, Asn, Ile, Leu, Lys, Met, Ser, Thr, Tyr, or Val; and Xaa₆ is Ala, Gln, Gly, Met, Ser, Tyr, or Val; and wherein the protease cleaves the peptide bond between Xaa₃ and Xaa₄ (SEQ ID NO:3).

In some embodiments, the positively-charged polymer may be polyethylenimine, polylysine, a cationic peptide, poly(dl-lactide-co-glycolide), poly(amidoamine), or poly(propylenimine). In an embodiment, the positively-charged polymer is polyethylenimine. In an embodiment, the polyethylenimine has a molecular weight from about 500 daltons to about 5000 daltons. In an embodiment, the polyethylenimine has an average molecular weight of about 1800 daltons. In an embodiment, the polyethylenimine has a branched structure.

In some embodiments, the phospholipid may be phosphatidic acid, phosphatidylethanolamine, phosphatidylcholine, phosphatidylserine, phosphatidylinositol, phosphotidylglycerol, or a sphingolipid. In some embodiments, the phospholipid comprises fatty acid side chains each having from 12-20 carbon atoms. In some embodiments, the fatty acid side chains are saturated, monounsaturated, diunsaturated, or triunsaturated. In some embodiments, the phospholipid is phosphtatidylethanolamine. In an embodiment, the phosphatidylethanolamine is 1,2-dioleoyl-sn-glycero-3-phosphoethanolamine.

In some embodiments, the covalent linkages may be peptide bonds, amide bonds, ester bonds, ether bonds, alkyl bonds, carbonyl bonds, alkenyl bonds, thioether bonds, disulfide bonds, and/or azide bonds. In some embodiments, each covalent linkages is a peptide bond.

In one aspect, the invention is a nanoparticle composition for delivery of a polynucleotide to a cell or tissue that overexpresses a protease, and the composition includes a plurality of protease-sensitive, polynucleotide-binding molecules suspended in an aqueous medium and aggregated to form one or more nanoparticles.

In some embodiments, the nanoparticle composition includes one or more polynucleotides that are non-covalently bound to the positively-charged polymers of the protease-sensitive, polynucleotide-binding molecule. In some embodiments, the polynucleotide(s) is single-stranded RNA, double-stranded RNA, single-stranded DNA, or double-stranded RNA. In some embodiments, the polynucleotide(s) is siRNA. In some embodiments, the polynucleotides are two or more different species of siRNA. In some embodiments, the polynucleotide is an antisense oligonucleotide. In some embodiments, the polynucleotide is an siRNA or antisense nucleotide suitable for treating cancer. In some embodiments, the polynucleotide targets the expression of one or more of survivin, Eg5, EGFR, XIAP, CDC45L, SUV420h1, WEE1, HDAC2, RBX 1, CDK4, CSN5, FOXM1, R1 (RAM2), LSD1, CSTF2, Nectin-4, ERCC6L, PKIB, NAALADL2, PRMT1, COPZ1, SYNGR4, P-glycoprotein, VEGFR, and VEGF.

In some embodiments, the nanoparticle composition has a nitrogen:phosphate ratio from about 1:5 to about 1:50.

In some embodiments, the nanoparticles are micelles. In some embodiments the micelles have an average diameter from about 10 to about 50 nm.

In some embodiments, the cell or tissue that overexpresses a protease is associated with cancer. In some embodiments, the cancer may be ovarian cancer, breast cancer, prostate cancer, uterine cancer, cervical cancer, prostate cancer, and melanoma, pancreatic cancer, tongue cancer, bladder cancer, carcinoma, gastric cancer, stomach cancer, liver cancer, hepatoma, colorectal cancer, lung cancer, gall bladder cancer, nasopharyngeal cancer, oral cancer, squamous cell cancer, kidney cancer, renal cancer, laryngeal cancer, leukemia, bone cancer, skin cancer, basal cell carcinoma, extra-gastrointestinal stromal cancer, or thyroid cancer.

In some embodiments, the peptide of the of protease-sensitive, polynucleotide-binding molecules is cleavable by a protease. In some embodiments, the protease is a matrix metalloproteinase. In some embodiments, the matrix metalloproteinase is MMP-2 and/or MMP-9. In some embodiments, cleavage of the peptide causes release of the uncharged hydrophilic polymers from the nanoparticles. In some embodiments, cleavage of the peptide results in increased cellular uptake of polynucleotides bound to the positively-charged polymers of the nanoparticles.

In some embodiments, the nanoparticle composition includes a hydrophobic pharmaceutical agent. In some embodiments, the hydrophobic pharmaceutical agent is an anti-cancer agent. In some embodiments, the anti-cancer agent may be altretamine, aminoglutethimide, amsacrine (m-AMSA), azacitidine, baccatin III, bleomycin, busulfan, carmustine (BCNU), chlorambucil, cytarabine HCl, dacarbazine, dactinomycin, daunorubicin, docetaxel, doxorubicin, etoposide (VP-16), 5-fluorouracil, floxuridine, flutamide, hydroxyurea, ifosfamide, leuprolide acetate, lomustine (CCNU), melphalan, methotrexate, mitomycin, mitotane (o.p′-DDD), octreotide, paclitaxel, pentostatin, plicamycin, procarbazine HCl, semustine (methyl-CCNU), streptozocin, tamoxifen citrate, teniposide (VM-26), thioguanine, thiotepa, vindesine, vinblastine, or vincristine sulfate. In some embodiments, the hydrophobic pharmaceutical agent is paclitaxel.

In some embodiments, the nanoparticle composition consists only of a plurality of protease-sensitive, polynucleotide-binding molecules.

In one aspect, the invention is a pharmaceutical composition that includes a nanoparticle composition of the invention suspended in an aqueous buffer.

In some embodiments, the pharmaceutical composition includes an excipient. For example, the excipient may be a buffer, electrolyte, or other inert component.

In one aspect, the invention is a method of making a protease-sensitive, polynucleotide-binding molecule from an uncharged hydrophilic polymer having a first reactive group, a peptide having a target cleavage site for a protease and having a second and a third reactive group, a positively-charged polymer having a fourth and a fifth reactive group, and a phospholipid having a sixth reactive group, the method including the steps of: reacting the first reactive group on the uncharged hydrophilic polymer with the second reactive group on the peptide, wherein the uncharged hydrophilic polymer and the peptide are present in about a 1:1 molar ratio, to create a covalent linkage between the uncharged hydrophilic polymer and the peptide; reacting the third reactive group on the peptide with the fourth reactive group on the positively-charged polymer, wherein the peptide and the positively-charged polymer are present in about a 1:1 molar ratio, to create a covalent linkage between the peptide and the positively-charged polymer; and reacting the fifth reactive group on the positively-charged polymer with the sixth reactive group on the phospholipid, wherein the positively-charged polymer and the phospholipid are present in about a 1:1 molar ratio, to create a covalent linkage between the positively-charged polymer and the phospholipid.

The steps of the method can be performed in any order. In one embodiment, the uncharged hydrophilic polymer and peptide are reacted first, the peptide and positively-charged polymer are reacted second, and the positively-charged polymer and phospholipid are reacted third. In one embodiment, the uncharged hydrophilic polymer and peptide are reacted first, the positively-charged polymer and phospholipid are reacted second, and the peptide and positively-charged polymer are reacted third. In one embodiment, the peptide and positively-charged polymer are reacted first, the uncharged hydrophilic polymer and peptide are reacted second, and the positively-charged polymer and phospholipid are reacted third. In one embodiment, the peptide and positively-charged polymer are reacted first, the positively-charged polymer and phospholipid are reacted second, and the uncharged hydrophilic polymer and peptide are reacted third. In one embodiment, the positively-charged polymer and phospholipid are reacted first, the uncharged hydrophilic polymer and peptide are reacted second, and the peptide and positively-charged polymer are reacted third. In one embodiment, the positively-charged polymer and phospholipid are reacted first, the peptide and positively-charged polymer are reacted second, and the uncharged hydrophilic polymer and peptide are reacted third.

In some embodiments, the uncharged hydrophilic polymer is polyethylene glycol 2000-N-hydroxysuccinamide ester.

In some embodiments, the some embodiments, the peptide comprises the amino acid sequence Gly-Pro-Leu-Gly-Ile-Ala-Gly-Gln (SEQ ID NO:1).

In some embodiments, the positively-charged polymer is branched polyethylenimine having an average molecular weight of about 1800 daltons.

In some embodiments, the phosphatidylethanolamine is 1,2-dioleoyl-sn-glycero-3-phosphoethanolamine-N-(glutaryl).

In some embodiments, the uncharged hydrophilic polymer and peptide are reacted by performing the steps of: reacting the peptide and polyethylene glycol 2000-N-hydroxysuccinimide ester in a 1.2:1 molar ratio in a carbonate-buffered aqueous solution at pH 8.2 under nitrogen protection at 4° C. to create a peptide-polyethlyne glycol product; and removing the unreacted peptide by dialysis against H₂O.

In some embodiments, the peptide and positively-charged polymer are reacted by performing the steps of: reacting a peptide-polyethylene glycol product with a 20-fold molar excess of N-(3-dimethylaminopropyl)N′-ethylcarbodiimide hydrochloride and N-hydroxysuccinimide to create activated peptide-polyethylene glycol product; reacting the activated peptide-polyethylene glycol product with a polyethylenimine-phosphoethanolamine product in a 1:1 molar ratio in the presence of a trace amount of triethylamine at room temperature to create a protease-sensitive, polynucleotide-binding molecule; and dialyzing the reaction product against H₂O.

In some embodiments, the positively-charged polymer and phospholipid are reacted by performing the steps of: reacting 1,2-dioleoyl-sn-glycero-3-phosphoethanolamine-N-(glutaryl) with a 20-fold molar excess of N-(3-dimethylaminopropyl)N′-ethylcarbodiimide hydrochloride and N-hydroxysuccinimide to create activated 1,2-dioleoyl-sn-glycero-3-phosphoethanolamine-N-(glutaryl); reacting the activated 1,2-dioleoyl-sn-glycero-3-phosphoethanolamine-N-(glutaryl) with branched polyethylenimine having an average molecular weight of about 1800 daltons at a 1:1 molar ratio in the presence of a trace amount of triethylamine at room temperature to create a polyethylenimine-phosphoethanolamine product; and removing the CHCl₃ by dialyzing the reaction product against H₂O.

In one aspect, the invention is a method of making a nanoparticle composition including the protease-sensitive, polynucleotide-binding molecule, the method including the steps of: providing a solution of the protease-sensitive, polynucleotide-binding molecule in a non-aqueous solvent; and replacing the non-aqueous solvent with an aqueous medium to form an aqueous suspension comprising nanoparticles, the nanoparticles comprising aggregates of a plurality of the protease-sensitive, polynucleotide-binding molecules.

The non-aqueous solvent may be replaced with an aqueous medium by any method. In some embodiments, the non-aqueous solvent is removed by dialyzing the solution of the protease-sensitive, polynucleotide-binding molecule against an aqueous medium. In some embodiments, the non-aqueous solvent is removed by evaporating the non-aqueous solvent to form a dry film of the protease-sensitive, polynucleotide-binding molecule and suspending the dry film of said molecule in an aqueous medium.

In some embodiments, the method includes the step of adding a hydrophobic pharmaceutical agent to the solution of the protease-sensitive, polynucleotide-binding molecule in a non-aqueous solvent, whereby the nanoparticles produced by replacing the non-aqueous solvent with an aqueous medium contain the hydrophobic pharmaceutical agent.

In some embodiments, the method includes the step of adding a hydrophobic pharmaceutical agent to the aqueous suspension of nanoparticles, whereby the hydrophobic pharmaceutical agent becomes incorporated into the nanoparticles.

In some embodiments, the method includes the step of adding a polynucleotide to the aqueous suspension of nanoparticles, whereby the polynucleotide becomes non-covalently bound to the positively-charged polymers of the nanoparticles. In some embodiments, two or more polynucleotides are added to the aqueous suspension and become bound to the positively-charged polymers of the nanoparticles.

In one aspect, the invention is a method of treating a disease or condition associated with a cell or tissue that overexpresses a protease, the method including administering to a subject having or suspected of having the disease or condition a nanoparticle composition of the invention.

In some embodiments, the disease or condition associated with a cell or tissue that overexpresses a protease is cancer.

In some embodiments, the nanoparticle composition is administered by a parenteral route. In some embodiments, the parenteral administration route is intravascular administration, peri- and intra-tissue administration, subcutaneous injection or deposition, subcutaneous infusion, intraocular administration, or direct application at or near a site of neovascularization.

In some embodiments, the nanoparticle comprises a polynucleotide. In some embodiments, the polynucleotide targets the expression of one or more of survivin, Eg5, EGFR, XIAP, CDC45L, SUV420h1, WEE1, HDAC2, RBX 1, CDK4, CSN5, FOXM1, R1 (RAM2), LSD1, CSTF2, Nectin-4, ERCC6L, PKIB, NAALADL2, PRMT1, COPZ1, SYNGR4, P-glycoprotein, VEGFR, and VEGF.

In some embodiments, the nanoparticle comprises a hydrophobic pharmaceutical agent. In some embodiments, the hydrophobic pharmaceutical agent is one or more of altretamine, aminoglutethimide, amsacrine (m-AMSA), azacitidine, baccatin III, bleomycin, busulfan, carmustine (BCNU), chlorambucil, cytarabine HCl, dacarbazine, dactinomycin, daunorubicin, docetaxel, doxorubicin, etoposide (VP-16), 5-fluorouracil, floxuridine, flutamide, hydroxyurea, ifosfamide, leuprolide acetate, lomustine (CCNU), melphalan, methotrexate, mitomycin, mitotane (o.p′-DDD), octreotide, paclitaxel, pentostatin, plicamycin, procarbazine HCl, semustine (methyl-CCNU), streptozocin, tamoxifen citrate, teniposide (VM-26), thioguanine, thiotepa, vindesine, vinblastine, and vincristine sulfate.

In one aspect, the invention is a kit for use in treating a disease or condition associated with a cell or tissue that overexpresses a protease, the kit including a protease-sensitive, polynucleotide-binding molecule of the invention and packaging therefor.

In some embodiments, the protease-sensitive, polynucleotide-binding molecule is provided as a dry powder or film. In some embodiments, the protease-sensitive, polynucleotide-binding molecule is provided in the form of an aqueous suspension containing a plurality of nanoparticles containing the protease-sensitive, polynucleotide-binding molecules.

In some embodiments, the kit includes a polynucleotide.

In some embodiments, the kit includes a hydrophobic pharmaceutical agent.

In some embodiments, the kit includes instructions for reconstituting the protease-sensitive, polynucleotide-binding molecule as micelles in an aqueous suspension. In some embodiments, the kit includes instructions for forming a nanoparticle composition containing the protease-sensitive, polynucleotide-binding molecule and a polynucleotide. In some embodiments, the kit includes instructions for forming a nanoparticle composition containing the protease-sensitive, polynucleotide-binding molecule and a hydrophobic pharmaceutical agent. In some embodiments, the kit includes instructions for use of the kit for treating a disease or condition associated with a cell or tissue that overexpresses a protease according to a method of the invention. In some embodiments, the kit includes instructions for forming non-covalent bonds between the polynucleotide and the nanoparticle composition.

In one aspect, the invention is a kit for use in treating a disease or condition associated with a cell or tissue that overexpresses a protease, the kit including a nanoparticle composition containing the protease-sensitive, polynucleotide-binding molecule of the invention and packaging therefor.

In one aspect, the invention is a kit for treating a disease or condition associated with a cell or tissue that overexpresses a protease, the kit including a pharmaceutical composition containing the protease-sensitive, polynucleotide-binding molecule of the invention and packaging therefor.

### BRIEF DESCRIPTION OF THE DRAWINGS

FIG. 1 is a schematic illustration of a molecule of the invention and its assembly with a small hydrophobic molecule and a polynucleotide to form a nanoparticle composition of the invention. Also shown is the de-shielding of the nanoparticle by cleavage of the protease-sensitive peptide to remove the hydrophilic polymer from the surface of the nanoparticle.

FIG. 2 shows a scheme for synthesis of PEG-pp-PEI-PE.

FIG. 3 is an ¹H NMR spectrum of a PEG-pp-PEI-PE in CDCl₃ (thick line) and D₂O (thin line).

FIG. 4A is graph showing pyrene fluorescence at various concentrations of PEG-pp-PEI-PE in a determination of the critical micelle concentration of PEG-pp-PEI-PE. FIG. 4B shows the particle size of PEG-pp-PEI-PE micelles at different pH values.

FIG. 5A shows a thin layer chromatograph showing cleavage of PEG-pp-PEI-PE after incubation with MMP-2. FIG. 5B shows fluorescence from Rh-PE incorporated into micelles that were analyzed by size exclusion-HPLC. The top panel shows PEI-PE micelles, the middle panel shows untreated PEG-pp-PEI-PE micelles, the lower panel shows PEG-pp-PEI-PE micelles treated with MMP-2. FIG. 5C shows the zeta potential of several micelle compositions. The top panel shows PEI-PE micelles, the second panel from the top shows PEI-PE micelles in the presence of PEG-peptide conjugate, the third panel from the top shows untreated PEG-pp-PEI-PE micelles, and the bottom panel shows PEG-pp-PEI-PE micelles treated with MMP-2.

FIG. 6 shows the complexation of siRNA by PEI 1800 Da, PEI-PE, and PEG-pp-PEI-PE. In this experiment, 0.4 μg of free siRNA or siRNA complexes were analyzed by gel electrophoresis on a 2% pre-cast agarose gel containing ethidium bromide.

FIG. 7 shows an RNase protection assay. The samples were incubated with Ambion RNase Cocktail®, followed by complex dissociation using dextran sulfate. Samples were then analyzed by gel electrophoresis.

FIG. 8 is graph of ethidium bromide fluorescence in the presence of siRNA bound to polymers at various N/P ratios. The siRNA complexes were incubated with 12 μg/mL of ethidium bromide and analyzed before and after dissociation with heparin at 10 units per μg of siRNA.

FIG. 9 is a graph showing in vitro release of paclitaxel from PEG-pp-PEI-PE/PTX/siRNA. The released PTX was measured by RP-HPLC after dialysis (cutoff 2,000 Da) against 1M sodium salicylate at 37° C.

FIG. 10A is graph showing the size of various particles as determined by dynamic light scattering. FIG. 10B is transmission electron micrograph of PEG-pp-PEI-PE/paclitaxel/siRNA particles. FIG. 10C is graph showing the size distribution of PEG-pp-PEI-PE/paclitaxel/siRNA particles. FIG. 10D shows the zeta potential of PEG-pp-PEI-PE/paclitaxel/siRNA particles. FIG. 10E shows the size distribution PEG-pp-PEI-PE/paclitaxel/siRNA particles after incubation in the presence of serum for various periods.

FIG. 11A shows in vitro cellular uptake of fluorescently labeled siRNA complexed with PEI-PE (b), untreated PEG-pp-PEI-PE (c), and MMP-2-cleaved PEG-pp-PEI-PE (d). Sample (a) had untreated cells. Data are shown as a plot of individual cells on left and summarized in bar graph on right. FIG. 11B shows in vitro cellular uptake after MMP-2 treatment of fluorescently labeled siRNA complexed with PEG-pp-PEI-PE containing an uncleavable peptide (b′), 25 kD PEI (c′), PEG-pp-PEI-PE (d′), and PEI-PE (e′). Sample (a′) had free fluorescent siRNA. FIG. 11C shows confocal microscopic images of the samples from FIG. 11B after 4h incubation in 10% FBS and staining with Hoechst 33342 and LysoTracker® Green DND-26.

FIG. 12A shows FACS analysis of A549 cells after incubation for 2 hours in complete medium with complexes containing Oregon green-paclitaxel and siGLO siRNA. Scatter plots show untreated cells (a), and cells treated with paclitaxel and siRNA complexed with 25 kD PEI (b), PEG-pp-PEI-PE containing an uncleavable peptide (c), and PEG-pp-PEI-PE (d). Bar graph on the right shows the relative levels of co-delivery siRNA and PTX into cells from (a), (c), and (d). FIG. 12B shows confocal microscopic images of the samples from FIG. 12A after staining with Hoechst 33342.

FIG. 13A is a graph showing relative expression of GFP in copGFP A549 cells after one (grey) or three (black) transfections with PEI-PE/siRNA (a), PEG-pp-PEI-PE/siRNA (b), PEG-pp-PEI-PE uncleavable/siRNA (c), 25 kD PEI/siRNA (d), and nothing (e). FIG. 13B shows confocal microscopic images of the samples from FIG. 13A as well as samples treated in parallel with scrambled siRNAs. Cells were stained with Hoechst 33342 to visualize nuclei. FIG. 13C is graph of levels of surviving protein analyzed by ELISA after incubation of A549 T24 cells with various concentrations PEG-pp-PEG-PE/anti-survivin siRNA for 48 h.

FIG. 14A are graphs of cell viability as determined by Cell Titer Blue® assay after cells were treated for 72 h with various concentrations of paclitaxel, PEG-pp-PEI-PE/PTX micelles, and PEG-pp-PEI-PE uncleavable/PTX micelles. Graph on the left shows A549 cells, and graph on the right shows A549 T24 cells. FIG. 14B is a graph of cell viability as determined by Cell Titer Blue® assay after A549 T24 cells were treated for 72 h with various concentrations of paclitaxel, PEG-pp-PEI-PE/PTX micelles, and PEG-pp-PEI-PE/PTX/siRNA micelles.

FIG. 15A shows delivery of paclitaxel and siRNA to various tissues in vivo. PEG-pp-PEI-PE complexed with Oregon green-PTX and siGLO siRNA (thick lines) or HBSS (thin lines) was injected into mice intravenously, and tissues were analyzed by FACS 2 h post-injection. Graphs in the first and third rows show fluorescence from paclitaxel, and graphs in the second and fourth rows show fluorescence from siRNA. Tissues analyzed were heart (left graphs, rows 1 and 2), liver (center graphs, rows 1 and 2), spleen (right graphs, rows 1 and 2), lung (left graphs, rows 3 and 4), kidney (center graphs, rows 3 and 4), and tumor (right graphs, rows 3 and 4). FIG. 15B shows scatter plots of tumor cells from mice treated with nothing (a), PEG-pp-PEI-PE uncleavable/PTX/siRNA (b), and PEG-pp-PEI-PE/PTX/siRNA (c).

### DETAILED DESCRIPTION OF THE INVENTION

The present invention provides compositions and methods for the delivery of a polynucleotide, hydrophobic pharmaceutical agent, or both to a cell or tissue that overexpresses a protease. The compositions and methods employ an amphipathic molecule that self-assembles into micellar nanoparticles. The micellar nanocarrier possesses several key features for delivery of polynucleotides and hydrophobic drugs, including (i) excellent stability; (ii) efficient condensation of polynucleotides by a positively-charged polymer; (iii) hydrophobic drug solubilization in the lipid “core”; (iv) passive tumor targeting via the enhanced permeability and retention (EPR) effect; (v) tumor targeting triggered by the protease-sensitive peptide; and (vi) enhanced cell internalization after protease-dependent exposure of the previously hidden positively-charged polymer. These cooperative functions ensure the improved tumor targetability, enhanced tumor cell internalization, and synergistic antitumor activity of co-loaded siRNA and drug.

A hydrophobic pharmaceutical agent for use in the invention is soluble in the core of nanoparticles of the invention, specifically in the lipid acyl chains found at the core.

An uncharged molecule or portion of a molecule as used herein is one that carries no net charge in an aqueous medium at physiological pH and temperature. A positively-charged molecule or portion of a molecule is one that has a net positive charge at physiological pH and temperature. A negatively-charged molecule or portion of a molecule is one that carries a net negative charge at physiological pH and temperature.

As used herein, “overexpress” and “overexpression” refer to a level of expression of a protein, for example, a protease, by a cell or tissue that is higher than the normal range of expression for that cell or tissue. Therefore, whether a protein, for example, a protease, is overexpressed depends on the type of cell or tissue, the level of expression, and other parameters of the cell or tissue in its physiological context. It is known in the art that overexpression of certain proteases by a cell or tissue is a phenotypic marker of cancers or precancerous conditions.

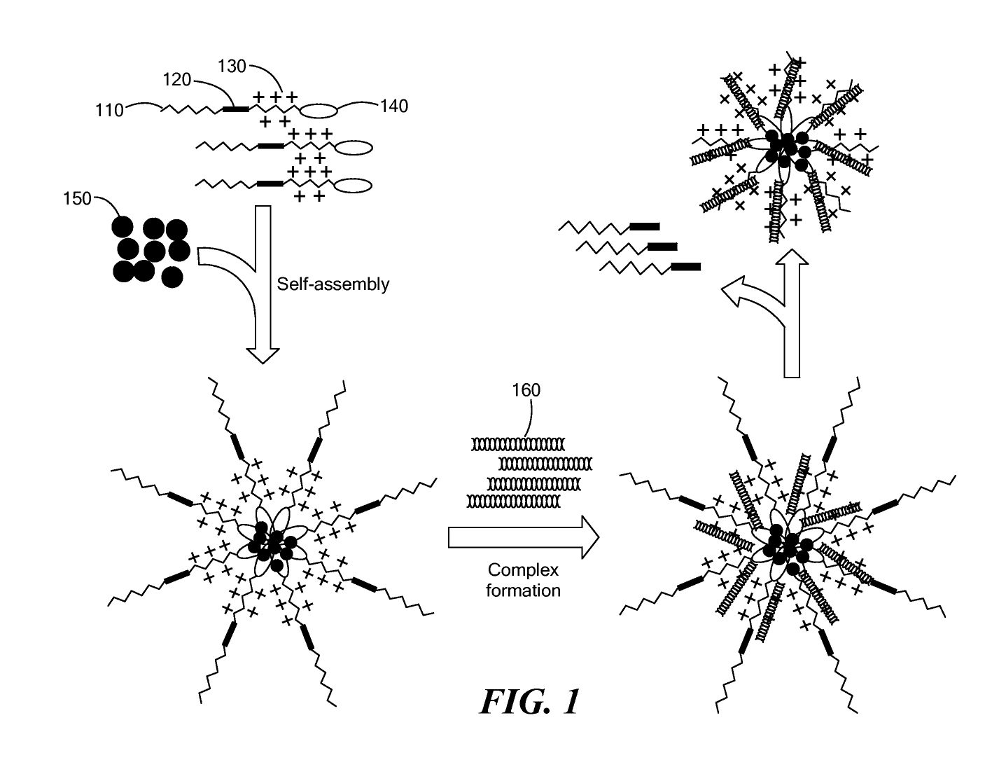

The invention includes a protease-sensitive, polynucleotide-binding molecule that can form micellar nanoparticles. As shown in FIG. 1, the molecule contains a series of covalent linkages between an uncharged hydrophilic polymer (110), a protease-sensitive peptide (120), a positively-charged polymer (130), and a phospholipid or other amphipathic moiety (140). Due to its amphipathic character, the molecule self-assembles into micellar nanoparticles. When nanoparticles assemble in the presence of a hydrophobic pharmaceutical agent (150), the hydrophobic pharmaceutical agent becomes incorporated into the nanoparticle's lipophilic core. Polynucleotides (160), having negatively-charged phosphate backbones, stably bind to the positively charged polymers in the micellar nanoparticles. Binding to the nanoparticles causes the polynucleotides to condense and become nuclease-resistant. The uncharged hydrophilic polymer forms the surface of the nanoparticle and shields the positively-charged polymer from other solutes. Highly charged nanoparticles are cleared from the circulation more rapidly, so the charge shielding provided by the uncharged polymer extends the blood circulation time of the nanoparticle. However, the charge shielding also impairs cellular uptake of nanoparticles and the cargo that they carry. This side effect is overcome by the protease-sensitive peptide, which is cleaved by a protease that recognizes a specific target sequence in the peptide. Cleavage of the protease-sensitive peptide results in the de-shielding of the nanoparticle and exposure of the positively-charged polymer, which facilitates cellular uptake of the nanoparticle. Consequently, the nanoparticle of the invention can preferentially deliver polynucleotides and/or hydrophobic pharmaceutical agents to a cell or tissue that overexpresses a protease that specifically cleaves the target sequence in the peptide.

The peptide may be any peptide that has an amino acid sequence that corresponds to the target cleavage site of a protease. For example, the target cleavage site may be specific for a matrix metalloproteinase. Many metalloproteinase substrates are known, and consensus target cleavage sites for matalloproteinases generally and for individual family members for have been described [26, 27]. Thus, the peptide may have an amino acid sequence identical to a matrix metalloproteinase cleavage site in a naturally-occurring protein substrate. For example, the peptide may have an amino acid sequence identical to the matrix metalloproteinase cleavage site from Aggrecan, Big endothelin-1, Brevican/BEHAB, Collagen-α1(I), Collagen-α1(X), Decorin, FGFR-1, Galectin-3, IGFBP-3, IL-1β, Laminin-5 γ2-chain, α2-Macroglobulin, MCP-3, Pregnancy zone protein, Pro-MMP-1, Pro-MMP-2, SPARC, Substance P, Betaglycan, Dentin, Integrin-αV, Integrin-α6, Integrin-αX, Integrin-α9, NG2 proteoglycan, Neurocan, or PAI-3. The peptide may include the sequence Gly-Pro-Leu-Gly-Ile-Ala-Gly-Gln (SEQ ID NO:1). Alternatively, the peptide may include an amino acid sequence identified as a matrix metalloproteinase cleavage in vitro. For example, the peptide may include the amino acid sequence Xaa₁-Xaa₂-Xaa₃-Xaa₄-Xaa₅-Xaa₆, wherein Xaa₁ is Ala, Ile, Pro, or Val; Xaa₂ is any amino acid; Xaa₃ is Ala, Asn, Gln, Glu, Gly Ser, or Thr; Xaa₄ is Arg, Ile, Leu, Met, Phe, or Tyr; Xaa₅ is any amino acid; Xaa₆ is Ala, Gln, Gly, Met, Ser, Tyr, or Val; and wherein the protease cleaves the peptide bond between Xaa₃ and Xaa₄ (SEQ ID NO:2). Alternatively, the peptide may include the amino acid sequence Xaa₁-Xaa₂-Xaa₃-Xaa₄-Xaa₅-Xaa₆, wherein Xaa₁ is Ala, Ile, Pro, or Val; Xaa₂ is Ala, Arg, Asn, Glu, Gly, Leu, Met, Phe, Tyr, or Val; Xaa₃ is Ala, Asn, Gln, Glu, Gly Ser, or Thr; Xaa₄ is Arg, Ile, Leu, Met, Phe, or Tyr; Xaa₅ is Ala, Arg, Asn, Ile, Leu, Lys, Met, Ser, Thr, Tyr, or Val; Xaa₆ is Ala, Gln, Gly, Met, Ser, Tyr, or Val; and wherein the protease cleaves the peptide bond between Xaa₃ and Xaa₄ (SEQ ID NO:3).

The peptide may be covalently linked the uncharged hydrophilic polymer and the positively-charged polymer through the amino and carboxyl groups at the ends of the peptide or through side chains. The uncharged, hydrophilic polymer and positively-charged polymer may be attached at or near the amino-terminus and carboxy-terminus, respectively, of the peptide. Alternatively, the uncharged, hydrophilic polymer and positively-charged polymer may be attached at or near the carboxy-terminus and amino-terminus, respectively, of the peptide.

The uncharged hydrophilic polymer may be any water-soluble polymer that is uncharged at physiological pH and temperature and has a flexible main chain. For example and without limitation, the uncharged hydrophilic polymer may be polyethylene glycol, polyvinylpyrrolidone, or polyacrylamide. If the uncharged hydrophilic polymer is polyethylene glycol, it may have an average molecular weight from about 1000 to about 10,000 daltons, from about 1000 to about 5000 daltons, from about 2000 to about 4000 daltons, or about 2000 daltons. The uncharged hydrophilic polymer may be a derivative of molecule described above. For example and without limitation, the uncharged hydrophilic polymer may be polyethylene glycol N-hydroxysuccinamide ester, or it may be another derivatized form of polyethylene glycol.

The positively-charged polymer may be any polymer that is positively charged at physiological pH and temperature. For example and without limitation, the positively-charged polymer may be polyethylenimine, polylysine, a cationic peptide, poly(dl-lactide-co-glycolide), poly(amidoamine), or poly(propylenimine). If the positively-charged polymer is polyethylenimine, it may have an average molecular weight from about 500 daltons to about 5000 daltons, from about 1000 to about 2000 daltons, from about 5000 to about 20,000 daltons, from about 20,000 to about 30,000 daltons, about 1800 daltons, or about 25,000 daltons. The polyethylenimine may have a linear structure, a branched structure, or a dendrimeric structure. The positively-charged polymer may be a derivative of molecule described above.

The phospholipid may be any stable phospholipid with amphipathic properties. For example and without limitation, the phospholipid may be phosphatidic acid, phosphatidylethanolamine, phosphatidylcholine, phosphatidylserine, phosphatidylinositol, phosphotidylglycerol, or a sphingolipid. The fatty acid chains in the phospholipid may be any length or structure that is compatible that allows the protease-sensitive, polynucleotide-binding molecule to form micelles. For example, the fatty acid chains may have from 9 to 20 carbon atoms, from 10 to 20 carbon atoms, from 12 to 20 carbon atoms, from 14 to 20 carbon atoms, from 16 to 20 carbon atoms, or from 8 to 24 carbons. The fatty acid chains in the phospholipid may be saturated, monounsaturated, diunsaturated, or triunsaturated. The unsaturated fatty acid side chains may have carbon-carbon double bonds in either a cis or trans configuration.

The covalent linkage may be any covalent bond that is stable at physiological pH and temperature. For example and without limitation, the covalent linkage may be a peptide bond, amide bond, ester bond, ether bond, alkyl bond, carbonyl bond, alkenyl bond, thioether bond, disulfide bond, or azide bond. The covalent linkage may be cyclical. For example and without limitation, the covalent linkage may be a 1,2,3-triazole or cyclohexene.

The micellar nanoparticles may assume various sizes and morphologies. For example and without limitation, they may be spherical or worm-like (i.e., long and flexible). The micellar nanoparticles may have an average diameter from about 10 nm to about 100 nm, from about 10 nm to about 50 nm, or from about 20 to about 40 nm. The micellar nanoparticles may consist only of the protease-sensitive, polynucleotide-binding molecule described herein.

Alternatively, the micellar nanoparticles may contain one or more polynucleotides non-covalently bound to the positively charged polymer of the protease-sensitive, polynucleotide-binding molecule. The polynucleotide may be of any type of nucleic acid molecule. For example, the polynucleotide may be a molecule of single-stranded RNA, double-stranded RNA, single-stranded DNA, or double-stranded RNA. The polynucleotide may be a molecule of siRNA. The polynucleotide may be an oligonucleotide. For example, the polynucleotide may be an antisense oligonucleotide. The polynucleotide may target a gene involved in cancer. For example, the polynucleotide may target survivin, Eg5, EGFR, XIAP, CDC45L, SUV420h1, WEE1, HDAC2, RBX 1, CDK4, CSN5, FOXM1, R1 (RAM2), LSD1, CSTF2, Nectin-4, ERCC6L, PKIB, NAALADL2, PRMT1, COPZ1, SYNGR4, P-glycoprotein, VEGFR, and/or VEGF. The micellar nanoparticles may have two or more different species of polynucleotides.

The micellar nanoparticles may be formed by adding the protease-sensitive, polynucleotide-binding molecule and the polynucleotide in a ratio that promotes condensation of the polynucleotide in the nanoparticle. For example, a micellar nanoparticle made by adding a protease-sensitive, polynucleotide-binding molecule having polyethylenimine as its positively-charged polymer and the polynucleotide in a nitrogen:phosphate ratio of about 1:1 to about 1:50, about 1:2 to about 1:50, about 1:5 to about 1:50, about 1:5 to about 1:25, about 1:10 to about 1:25. The degree of condensation may be assess by change in diameter of nanoparticle size, by protection of the polynucleotide from nuclease digestion, or by other methods.

The micellar nanoparticles may contain one or more hydrophobic pharmaceutical agents. The hydrophobic pharmaceutical agent may be any hydrophobic compound that can be used to treat a disease or condition. For example, the hydrophobic pharmaceutical agent may be an anti-cancer agent. For example, the hydrophobic pharmaceutical agent may be altretamine, aminoglutethimide, amsacrine (m-AMSA), azacitidine, baccatin III, bleomycin, busulfan, carmustine (BCNU), chlorambucil, cytarabine HCl, dacarbazine, dactinomycin, daunorubicin, docetaxel, doxorubicin, etoposide (VP-16), 5-fluorouracil, floxuridine, flutamide, hydroxyurea, ifosfamide, leuprolide acetate, lomustine (CCNU), melphalan, methotrexate, mitomycin, mitotane (o.p′-DDD), octreotide, paclitaxel, pentostatin, plicamycin, procarbazine HCl, semustine (methyl-CCNU), streptozocin, tamoxifen citrate, teniposide (VM-26), thioguanine, thiotepa, vindesine, vinblastine, vincristine sulfate, or any combination thereof. The hydrophobic pharmaceutical agent may be a small molecule drug having a molecular weight of less than 2000 daltons, less than 1500 daltons, less than 1000 daltons, or less than 500 daltons.

The cell or tissue that overexpresses a protease may be associated with a disease or condition. For example, the cell or tissue that overexpresses a protease may be associated with cancer. For example, the cell or tissue that overexpresses a protease may be associated with ovarian cancer, breast cancer, prostate cancer, uterine cancer, cervical cancer, prostate cancer, and melanoma, pancreatic cancer, tongue cancer, bladder cancer, carcinoma, gastric cancer, stomach cancer, liver cancer, hepatoma, colorectal cancer, lung cancer, gall bladder cancer, nasopharyngeal cancer, oral cancer, squamous cell cancer, kidney cancer, renal cancer, laryngeal cancer, leukemia, bone cancer, skin cancer, basal cell carcinoma, extra-gastrointestinal stromal cancer, or thyroid cancer.

The protease-sensitive peptide within the micellar nanoparticle is cleavable in the presence of a protease specific for the target cleavage site in the peptide. The protease-sensitive peptide covalently links the uncharged polymer to the rest of the protease-sensitive, polynucleotide-binding molecule. Consequently, cleavage of the protease-sensitive peptide in the presence of a cell or tissue that overexpresses the specific protease results in release of the uncharged hydrophilic polymers from the nanoparticles. The uncharged hydrophilic polymers shield the charge of the nanoparticle from the aqueous environment, and protease-dependent cleavage of the molecule causes the charge of the nanoparticle to become deshielded. The deshielding of the nanoparticle's charge promotes cellular uptake of the nanoparticle (FIG. 1). Thus, when the nanoparticle contains one more bound polynucleotides and hydrophobic pharmaceutical agents, cleavage of the protease-sensitive peptide increases the cellular uptake of these components as well. In addition, the protease-dependent deshielding of the nanoparticle facilitates release of the polynucleotide(s) and/or hydrophobic pharmaceutical agent(s) from an intracellular vesicular compartment into the cytoplasm.

The micellar nanoparticle may be suspended in an aqueous medium for use or storage. The aqueous medium may contain excipients to promote the stability of the nanoparticles or their effectiveness in delivery of polynucleotides and/or hydrophobic pharmaceutical agents. Such excipients are well known in the art. For example and without limitation, the suspension of micellar nanoparticles may contain one or more buffers, electrolytes, agents to prevent aggregation of nanoparticles, agents to prevent adherence of nanoparticles to the surfaces of containers, cryoprotectants, and/or pH indicators.

The invention includes methods of making the protease-sensitive, polynucleotide-binding molecules of the invention from the individual chemical components. One step of the method entails reacting a reactive group on the uncharged hydrophilic polymer with a reactive group on the protease-sensitive peptide to form a covalent linkage between these two components. In another step, a reactive group on the protease-sensitive peptide is reacted with a reactive group on the positively-charged polymer to form a covalent linkage between these two components. In another step, a reactive group on the positively-charged polymer is reacted with a reactive group on the phospholipid to form a covalent linkage between these two components.

The steps required to make the protease-sensitive, polynucleotide-binding molecules of the invention can be performed in any order. For example, the uncharged hydrophilic polymer and protease-sensitive peptide can be joined first, the protease-sensitive peptide and positively-charged polymer can be joined second, and the positively-charged polymer and phospholipid can be joined third. Alternatively, the uncharged hydrophilic polymer and protease-sensitive peptide can be joined first, and the positively-charged polymer and phospholipid can be joined second, and the protease-sensitive peptide and positively-charged polymer can be joined third. Alternatively, the protease-sensitive peptide and positively-charged polymer can be joined first, the uncharged hydrophilic polymer and protease-sensitive peptide can be joined second, and the positively-charged polymer and phospholipid can be joined third. Alternatively, the protease-sensitive peptide and positively-charged polymer can be joined first, the positively-charged polymer and phospholipid can be joined second, and the uncharged hydrophilic polymer and protease-sensitive peptide can be joined third. Alternatively, the positively-charged polymer and phospholipid can be joined first, the uncharged hydrophilic polymer and protease-sensitive peptide can be joined second, and the protease-sensitive peptide and positively-charged polymer can be joined third. Alternatively, the positively-charged polymer and phospholipid can be joined first, the protease-sensitive peptide and positively-charged polymer can be joined second, and the uncharged hydrophilic polymer and protease-sensitive peptide can be joined third. It will be understood by one of ordinary skill in the art that particular starting reactants of the reaction in each step of the method will vary depending on the sequence in which the steps are performed. Therefore, the starting reagents may be the individual components described above, or they may composite molecules consisting of two or three of the individual components described above that have been covalently linked according to the manner required by an earlier step of the method.

The individual steps of the method are performed to give products that have each of the starting reactants combined in a 1:1 molar ratio. The starting reactants may be present in a 1:1 molar ratio or in unequal molar amounts. Chemical reactions may be performed in organic solvents or in aqueous media. In addition to the reactants and solvents, the reactions may contain additional components as catalysts, solubilizers, and the like. For example, and without limitation, the reactions may include N-(3-dimethylaminopropyl)N′-ethylcarbodiimide hydrochloride, N-hydroxysuccinimide, pyridine, 4-dimethylaminopyridine, and/or triethylamine.

Each step of the method may be performed in a single step or in a series of sub-steps. A sub-step may entail a chemical reaction, an analytical method, a purification method, an exchange of solvent or medium, or any other process necessary to complete a step of the method. For example, the uncharged hydrophilic polymer and protease-sensitive peptide can be joined by: reacting the peptide and polyethylene glycol 2000-N-hydroxysuccinimide ester in a 1.2:1 molar ratio in a carbonate-buffered aqueous solution at pH 8.2 under nitrogen protection at 4° C. to create a peptide-polyethlyne glycol product; and removing the unreacted peptide by dialysis against H₂O. For example, the protease-sensitive peptide and positively-charged polymer can be joined by: reacting the product resulting from covalently linking the protease-sensitive peptide and polyethylene glycol with a 20-fold molar excess of N-(3-dimethylaminopropyl)N′-ethylcarbodiimide hydrochloride and N-hydroxysuccinimide to create activated peptide-polyethylene glycol product; reacting the activated peptide-polyethylene glycol product from with the product resulting from covalent linkage of polyethylenimine and phosphoethanolamine in a 1:1 molar ratio in CHCl₃ in the presence of a trace amount of triethylamine at room temperature to create a protease-sensitive, polynucleotide-binding molecule; and removing the CHCl₃ by dialyzing the product of the reaction in (b) against H₂O. For example, the positively-charged polymer and phospholipid can be joined by: reacting 1,2-dioleoyl-sn-glycero-3-phosphoethanolamine-N-(glutaryl) with a 20-fold molar excess of N-(3-dimethylaminopropyl)N′-ethylcarbodiimide hydrochloride and N-hydroxysuccinimide to create activated 1,2-dioleoyl-sn-glycero-3-phosphoethanolamine-N-(glutaryl); reacting the activated 1,2-dioleoyl-sn-glycero-3-phosphoethanolamine-N-(glutaryl) product with branched polyethylenimine having an average molecular weight of about 1800 daltons at a 1:1 molar ratio in CHCl₃ in the presence of a trace amount of triethylamine at room temperature to create a polyethylenimine-phosphoethanolamine product; and removing the CHCl₃ by dialyzing the reaction against H₂O.

The reactants react via reactive groups. The reactive groups allow formation of specific covalent linkages between two reactants. The reactive groups may be inherent in the starting components, the reactive groups may be added by derivatizing the starting components prior to performing the reaction in which the desired covalent linkage is formed. A reactant may have a single reactive group of a particular species, which directs formation of particular covalent linkage to a specific site within the reactant. Therefore, the protease-sensitive, polynucleotide-binding molecules of the invention can be made with one or more of the components having a specific orientation within the molecule. Alternatively, a reactant may have multiple reactive groups of a particular species, which allows formation of particular covalent linkage at multiple sites within the reactant. A reactant may have multiple species of reactive groups, thereby allowing formation of multiple different types of covalent linkages at distinct sites within the reactant. Therefore, the protease-sensitive, polynucleotide-binding molecules of the invention can be made with one or more of the components having a varied orientation within the molecule. For example, and without limitation, the reactive group may be a thiol, dithiol, trithiol, acyl, amine, carboxylic acid, azide, alkene, maleimide, alcohol, alkyne, dienyl, phenol, ester, or N-glutaryl. The reactive group may be joined to the reactant via a linker, for example, an oligoethylene glycol chain.

The invention includes methods of making micellar nanoparticles containing the protease-sensitive, polynucleotide-binding molecules of the invention. The method entails providing a solution of the protease-sensitive, polynucleotide-binding molecule in an organic solvent and replacing the non-aqueous solvent with an aqueous medium to form an aqueous suspension comprising nanoparticles made up of the molecule. The organic solvent may be replaced by an aqueous medium by any method known in the art. For example, the organic solution of the protease-sensitive, polynucleotide-binding molecule may be dialyzed against an aqueous medium to remove the organic solvent. Alternatively, the organic solvent may be evaporated to form a dry film of the protease-sensitive, polynucleotide-binding molecule, which is then resuspended in an aqueous medium.

The methods of making micellar nanoparticles containing the protease-sensitive, polynucleotide-binding molecules of the invention may include addition of other components. For example, a hydrophobic pharmaceutical agent may be included. One or more hydrophobic pharmaceutical agent may be added to the organic solution containing the protease-sensitive, polynucleotide-binding molecule, resulting in formation of micellar nanoparticles that contain the hydrophobic pharmaceutical agent(s). Alternatively, one or more hydrophobic pharmaceutical agents may be added to the aqueous suspension of micellar nanoparticles so that the hydrophobic pharmaceutical agent(s) is incorporated into the hydrophobic core of the nanoparticles. In another example, one or more polynucleotide(s) may be added to the aqueous suspension of micellar nanoparticles so that the polynucleotide(s) becomes non-covalently bound to the positively-charged polymer of the nanoparticle.

The invention includes methods of treating a disease or condition associated with a cell or tissue that overexpresses a protease by administering a composition of the micellar nanoparticles of the invention to a subject having or suspected of having the disease or condition. The nanoparticle composition may be administered by a parenteral route. For example, the nanoparticle composition may be administered by intravascular administration, peri- and intra-tissue administration, subcutaneous injection or deposition, subcutaneous infusion, intraocular administration, and direct application at or near a site of neovascularization.

The invention also includes kits for use in treating a disease or condition associated with a cell or tissue that overexpresses a protease. The kits may include a protease-sensitive, polynucleotide-binding molecule of the invention. The protease-sensitive, polynucleotide-binding molecule may be provided as a powder or dry film. The kit may include instructions for reconstituting the powder or dry film of protease-sensitive, polynucleotide-binding molecule as micellar nanoparticles in an aqueous suspension. Alternatively, the protease-sensitive, polynucleotide-binding molecule may be provided as micellar nanoparticles in an aqueous suspension.

The kit may include micellar nanoparticles of the invention. The micellar nanoparticles may consist only of the protease-sensitive, polynucleotide-binding molecule of the invention. Alternatively, the micellar nanoparticles may also include other components. For example, the micellar nanoparticles may also include a polynucleotide and/or a hydrophobic pharmaceutical agent.

The kit may include a pharmaceutical composition of the invention that includes a suspension of micellar nanoparticles containing a protease-sensitive, polynucleotide-binding molecule.

The kit may also include other components in separate containers. For example, the kit may include a polynucleotide and/or a hydrophobic pharmaceutical agent.

The kit may also include instructions for preparing and using the compositions of the invention. For example, the kit may include instructions for forming a nanoparticle composition containing the protease-sensitive, polynucleotide-binding molecule of the invention and a polynucleotide and/or hydrophobic pharmaceutical agent. The kit may include instructions for forming non-covalent bonds between a polynucleotide and a micellar nanoparticle of the invention. The kit may include instruction for incorporating a hydrophobic pharmaceutical agent into a micellar nanoparticle of the invention. The kit may include instructions for use of the kit in treating a disease or condition associated with a cell or tissue that overexpresses protease according to a method of the invention.

### EXAMPLES

### Example 1

### Materials and Methods

Materials. Polyethylene glycol 2000-N-hydroxysuccinimide ester (PEG2000-NHS) was purchased from Laysan Bio, Inc. (Arab, AL). 1,2-dioleoyl-sn-glycero-3-phosphoethanolamine (DOPE), 1,2-dioleoylsn-glycero-3-phosphoethanolamine-N-(lissamine rhodamine B sulfonyl) (ammonium salt) (Rh-PE), and 1,2-dioleoyl-snglycero-3-phosphoethanolamine-N-(glutaryl) (Glutaryl-PE) were purchased from Avanti Polar Lipids, Inc. (Alabaster, Ala.). Branched polyethylenimine (PEI) with a molecular weight of 1800 and 25,000 Da were purchased from Polysciences, Inc (Warrington, Pa.). The BCA Protein Assay Reagent, N-hydroxysuccinimide (NHS), chloroform, dichloromethane (DCM) and methanol were purchased from Thermo Fisher Scientific (Rockford, Ill.). Ninhydrin Spray reagent, Molybdenum Blue Spray reagent, heparin sodium salt, and 1-Ethyl-3-[3-dimethylaminopropyl] carbodiimide hydrochloride (EDC) were purchased from Sigma-Aldrich Chemicals (St. Louis, Mo.). Human active MMP2 protein (MW 66,000 Da) and TLC plate (silica gel 60 F254) were from EMD Biosciences (La Jolla, Calif.). Dialysis tubing (MWCO 2000 Da) was purchased from Spectrum Laboratories, Inc. (Houston, Tex.). Dulbecco's modified Eagle's medium (DMEM), penicillin streptomycin solution (PS) (100×), Hoechst 33342, LysoTracker®, Green DND-26 and trypsin-EDTA were from Invitrogen Corporation (Carlsbad, Calif.). FBS was purchased from Atlanta Biologicals (Lawrenceville, Ga.). SDS-PAGE pre-cast gel (4-20%) was purchased from Expedeon Ltd. (San Diego, Calif.). Ready Gel Zymogram Gel (10% polyacrylamide gel with gelatin), Zymogram Renaturation Buffer and Zymogram Development Buffer were purchased form Bio-Rad (Hercules, Calif.). Human non-small cell lung cancer A549 cells were from ATCC (Manassas, Va.). A549 cells stably expressing copGFP were from Cell Biolabs (San Diego, Calif.). Hank's Balanced Salt Solution (HBSS) was from Mediatech (Manassas, Va.). Ambion® RNase Cocktail® was purchased from Life Technologies (Grand Island, N.Y.). Ethidium bromide was from ICN Biomedicals (Aurora, Ohio). The MMP2-cleavable (GPLGIAGQ) and uncleavable (GGGPALIQ) octapeptides were synthesized by the Tufts University Core Facility (Boston, Mass.).

The anti-GFP siRNA (5′-AAGUGGUAGAAGCCGUAGCdTdT-3′ antisense) (SEQ ID NO:4), the scramble siRNA (5′-CCGUATCGUAAGCAGTACTdTdT-3′ antisense) (SEQ ID NO:5) and anti-survivin siRNA (5′-AGCGCAACCGGACGAAUCCdTdT-3′ antisense) (SEQ ID NO:6) were synthesized by Invitrogen.

The human non-small cell lung cancer (A549) cells, GFP expressing (copGFP A549) cells or cervical cancer (HeLa) cells were grown in complete growth media (DMEM supplemented with 50 U/mL penicillin, 50 mg/mL streptomycin and 10% FBS) at 37° C. at 5% CO2. The pacilitaxel resistant non-small cell lung cancer (A549 T24) cells (kindly provided by Dr. Susan Horwitz, Albert Einstein College of Medicine, Bronx, N.Y.) were maintained in the complete growth media containing 24 nM paclitaxel.

Synthesis, Purification and Characterization of PEG-pp-PEI-PE.

Three steps are involved in the synthesis of PEG-pp-PEI-PE. First, glutaryl-PE was activated with 20-fold molar excess of NHS/EDC for 2 h, then reacted with branched PEI (1,800 Da) (1:1, molar ratio) in chloroform in the presence of a trace amount of triethylamine at room temperature overnight [6]. The product PEI-PE was purified by dialysis (MWCO 3500 Da) against water for 48 h and characterized by ¹H NMR using D₂O and CDCl₃ as solvents.

Second, the MMP2-cleavable octapeptide (GPLGIAGQ) and PEG2000-NHS (1.2:1, molar ratio) were mixed and stirred in the carbonate buffer (pH 8.2) under nitrogen protection at 4° C. overnight. The unreacted peptide was removed by dialysis (MWCO 2000 Da) against distilled water. The product PEG2000-peptide (PEG-pp) was checked by RP-HPLC as described in a previous study [12].

Finally, PEG-pp was activated with NHS/EDC and reacted with PEI-PE (1:1, molar ratio) in the presence of triethylamine at room temperature overnight. The reaction mixture was dialyzed against water (MWCO 3500 Da) for 48 h. The PEG-pp-PEI-PE was characterized by ¹H NMR using D₂O and CDCl₃ as solvents. For synthesis of the uncleavable conjugate, the scramble peptide (GGGPALIQ) was used.

Particle Size, Zeta Potential and Morphology.

The particle size of PEG-pp-PEI-PE micelles, PEG-pp-PEI-PE/siRNA, or PEG-pp-PEI-PE/PTX/siRNA was measured by dynamic light scattering (DLS) on a Coulter® N4-Plus Submicron Particle Sizer (Beckman Coulter). The zeta potential was measured in HBSS by Zeta Potentiometer (Brookhaven Instruments). The morphology was analyzed by transmission electron microscopy (TEM) (model XR-41B) (Advanced Microscopy Techniques, Danvers, Mass.) using negative staining with 1% phosphotungstic acid (PTA).

Determination of Critical Micelle Concentration (CMC).

The CMC was determined by fluorescence spectroscopy using pyrene as a hydrophobic fluorescent probe [13]. Briefly, pyrene chloroform solution was added to the testing tube at the final concentration of 8×10⁻⁵ M and dried on a freeze-dryer overnight. Then, the polymers in HBSS were added to the tubes at a 5-fold serial dilution (from 10⁻¹ to 10⁻⁸ mg/mL) and incubated with shaking at room temperature for 24 h before measurement. The fluorescence intensity was measured on an F-2000 fluorescence spectrometer (Hitachi, Japan) with the excitation wavelengths (λₑₓ) of 337 nm (I3) and 334 nm (I1) and an emission wavelength (λₑₘ) of 390 nm. The intensity ratio (I337/I334) was calculated and plotted against the logarithm of the polymer concentration. The CMC value was obtained as the crossover point of the two tangents of the curves.

Cleavage Study of PEG-pp-PEI-PE by Human MMP2.

One mg/mL of the polymer was incubated with active human MMP2 (5 ng/mL) in pH 7.4 HBS containing 10 mM CaCl2 at 37° C. overnight [13]. Three methods were used to analyze the cleavage of the peptide linker. For thin layer chromatography (TLC), the samples were run in chloroform/methanol (8:2, v/v) followed by Dragendorff's reagent staining. For size exclusion chromatography, the polymers and Rh-PE were dissolved in chloroform and dried to form a thin film. The Rh-PE was incorporated into the polymeric micelles via hydration with HBSS as an indicator for the micelle peak in chromatograms. After enzymatic digestion, the reaction mixture was applied on a Shodex KW-804 size exclusion column at the flow rate of 1 mL/min of water and detected by both UV (280 nm) and fluorescence detectors (λₑₓ=570 nm, λₑₘ=595 nm) on a Hitachi HPLC system. The zeta potential of samples was also measured in HBSS to indicate the change of the charge.

Preparation of PEG-pp-PEI-PE/siRNA and PEG-pp-PEI-PE/PTX/siRNA Complexes.

To prepare PEG-pp-PEI-PE/siRNA complexes, siRNA was mixed with PEG-pp-PEI-PE micelles in HBSS at various N/P ratios and incubated at room temperature for 20 min, allowing for siRNA complex formation. For co-loading of PTX and siRNA, PTX and PEG-pp-PEI-PE were dissolved in chloroform and dried to form the drug-polymer film, followed by hydration with HBSS using vortex. The unentrapped PTX was removed by filtration through a 0.45 mm filter (GE Healthcare) [7]. The PTX in filtrate was measured on a reversed-phase C18 column (250 mm 4.6 mm) using an isocratic mobile phase of acetonitrile and water (60:40, v/v) at a flow rate of 1.0 mL/min and detected at UV 227 nm on a Hitachi HPLC system. The PTX-loaded micelles were incubated with siRNA in HBSS at room temperature for 20 min. Then, the particle size, zeta potential and morphology of the complexes were analyzed.

Gel Retardation Assay.

To confirm the siRNA complex formation, 0.4 mg of free siRNA or siRNA complexes (N/P 10, 20 and 40) were applied on a 2% pre-cast agarose gel containing ethidium bromide. The gel was run on an E-Gel® system (Invitrogen) for 15 min.

RNase Protection Assay.

The resistance to nuclease digestion was determined using an RNase protection assay. The samples containing 0.4 mg of siRNA were incubated with 0.48 units of Ambion RNase Cocktail® for 2 h at 37° C. The RNase was then inactivated by 20 mM EDTA before complex dissociation using 2 mg/mL of dextran sulfate (500 kDa) for 20 min at 37° C. Then, samples were analyzed on a 2% pre-cast agarose gel containing ethidium bromide.

Ethidium Bromide Exclusion Assay.

The siRNA complexes were incubated with 12 mg/mL of ethidium bromide. The recovery of siRNA from their complexes was assessed by the fluorescence intensity after dissociation with heparin at 10 units per mg of siRNA. The fluorescence intensity was measured at λₑₓ=530 nm and λₑₘ=640 nm on a microplate reader (Synergy HT, Biotek).

Protein Adsorption/Interaction.

To evaluate the blood protein adsorption/interaction, the nanoparticles (PEGpp-PEI-PE/PTX/siRNA) were incubated with the normal mouse serum (1:10, v/v) at 37° C. for 12 h. The particle size was analyzed by DLS on a Coulter® N4-Plus Submicron Particle Sizer.

In Vitro Drug Release.

The PTX release rate from the PEG-pp-PEI-PE/PTX/siRNA was studied by a dialysis method. Briefly, the PEG-pp-PEI-PE/PTX/siRNA (0.4 mL) was dialyzed (MWCO 2000 Da) against 40 mL of water containing 1 M sodium salicylate to maintain the sink condition [13] at 37° C. The PTX in the outside media was determined by RP-HPLC during the experiment.

In Vitro Cellular Uptake.

To study the cellular uptake, A549 cells were seeded in 24-well plates at 1.6×10⁵ cells/well 24 h before experiments. To study the influence of the MMP2 on the cellular uptake, the FAM-siRNA was used to prepare the siRNA polyplexes at N/P 40. The cells were washed and replaced with serum-free media. The siRNA polyplexes were added to cell media and incubated with cells for 1 h. To test the cellular uptake of the siRNA polyplexes without MMP2 pretreatment, the siGLO siRNA was used as an indicator. The siGLO siRNA polyplexes (N/P40) were incubated with cells in complete growth media for 4 h. To evaluate the in vitro co-delivery efficiency, the Oregon green PTX and siGLO siRNA were used to prepare the nanoparticles. The nanoparticles were incubated with the cells in complete growth media for 2 h.

Then, the media was removed and the cells were washed with serum-free media three times. For FACS analysis, the cells were trypsinized and collected by centrifugation at 2000 rpm for 4 min. After washing with ice-cold PBS, the cells were resuspended in 400 mL of PBS and applied on a BD FACS Calibur flow cytometer (BD Biosciences). The cells were gated upon acquisition using forward vs. side scatter to exclude debris and dead cells. The data was collected (10,000 cell counts) and analyzed with BD Cell Quest Pro Software. For confocal microscopy, the cells were fixed by 4% paraformaldehyde (PFA). To visualize cell nuclei, cells were stained with 5 mM of Hoechst 33342 for 15 min at RT. To indicate the endosome, cells were stained with LysoTracker® Green DND-26. The photos were taken with a Zeiss LSM 700 confocal microscope system at 63× magnification and analyzed using Zeiss Image Browser software.

Gene Down-Regulation.

The copGFP A549 or A549 T24 cells were seeded at 5×10⁴ cells/well in 24 well culture plates 24 h before transfection. The anti-GFP siRNA polyplexes (N/P40) were incubated with copGFP A549 cells in complete growth media for 48 h (one transfection) or for 3 transfections (every other day). The cells were collected and analyzed by flow cytometry. After 3 transfections, the cells were also pictured by confocal microscopy. To down-regulate the survivin protein, the PEG-pp-PEG-PE/anti-survivin siRNA complexes were incubated with A549 T24 cells for 48 h. Then the cells were collected and lysed. The total survivin in cell lysates was determined by a human total survivin immunoassay kit and normalized by the total protein concentration determined by the BCA protein assay.

Cytotoxicity Study.

To study the toxicity of the polymers, A549 or HeLa cells were seeded at 4000 cells/well in 96-well plates 24 h before treatments. A series of diluted polymer (PEI 1800 Da or PEG-pp-PEI-PE) solutions were added to cells and incubated for 72 h. To study the toxicity of the siRNA polyplexes, the siRNA polyplexes with various N/P ratios were added to cells and incubated for 72 h.

To study the cytotoxicity of PTX, PEG-pp-PEI-PE/PTX or PEG-pp-PEI PE/PTX/siRNA, A549 or A549 T24 cells were seeded at 2000 cells/well in 96-well plates 24 h before treatments. The PTX or its formulations were incubated with the cells for 72 h in complete growth media. The cell viability was determined by Cell Titer-Blue® Cell Viability Assay. Briefly, 15 mL of CellTiter-Blue® Reagent was diluted with 100 mL of complete growth medium per well and incubated with treated cells at 37° C. for 2 h. Thereafter, the fluorescence intensity was recorded at λₑₓ=560 nm and λₑₘ=590 nm using a Labsystems Multiskan MCC/340 microplate reader.

In Vivo Co-Delivery of PTX and siRNA.

Female nude mice (NU/NU, 4e6 weeks old) were purchased from Charles River laboratories (Wilmington, Mass.). All animal procedures were performed according to an animal care protocol approved by Northeastern University Institutional Animal Care and Use Committee. Approximately 5×10⁶ A549 cells suspended in 50 ml HBSS were mixed with the phenol-red free high concentration Matrigel® (1:1, v/v) and inoculated in nude mice by subcutaneous injection over their right flanks. The tumor was monitored for length (l) and width (w) by caliper and calculated by the equation V=lw²/2.

HBSS, the MMP2-sensitive PEG-pp-PEI-PE/Oregon green-PTX/siGLO siRNA complexes and their nonsensitive counterparts were intravenously injected in tumor-bearing mice with a tumor size of about 400 mm3 via tail vein. At 2 h post-injection, mice were anesthetized and sacrificed. The tumor and major organs (heart, liver, spleen, lung, and kidney) were collected. The fresh tissues were minced into small pieces and incubated in 400 U/mL of collagenase D solution for 30 min at 37° C. to dissociate cells [14]. The single-cell suspension was analyzed immediately by FACS.

Statistical Analysis.

Data were presented as mean±standard deviation (SD). The difference between the groups was analyzed using a one-way ANOVA analysis by the commercial software PASW® Statistics 18 (SPSS). P<0.05 was considered statistically significant.

### Example 2

### Synthesis and Characterization of PEG-Pp-PEI-PE

In this study, to deliver siRNA and hydrophobic drugs, a simple but multifunctional micellar nanocarrier constructed by an MMP2-sensitive self-assembling copolymer, polyethylene glycol-peptide-polyethylenimine-1,2-dioleoyl-snglycero-3-phosphoethanolamine (PEG-pp-PEI-PE), was developed (FIG. 1). The MMP2-sensitive multifunctional micelles formed by the PEG-pp-PEI-PE conjugate were evaluated for co-delivery of siRNA and hydrophobic drugs in terms of their chemical and physicochemical properties, in vitro siRNA and drug delivery/codelivery efficiency, in vitro gene down-regulation and anticancer activity, and in vivo co-delivery efficiency and tumor targeting.

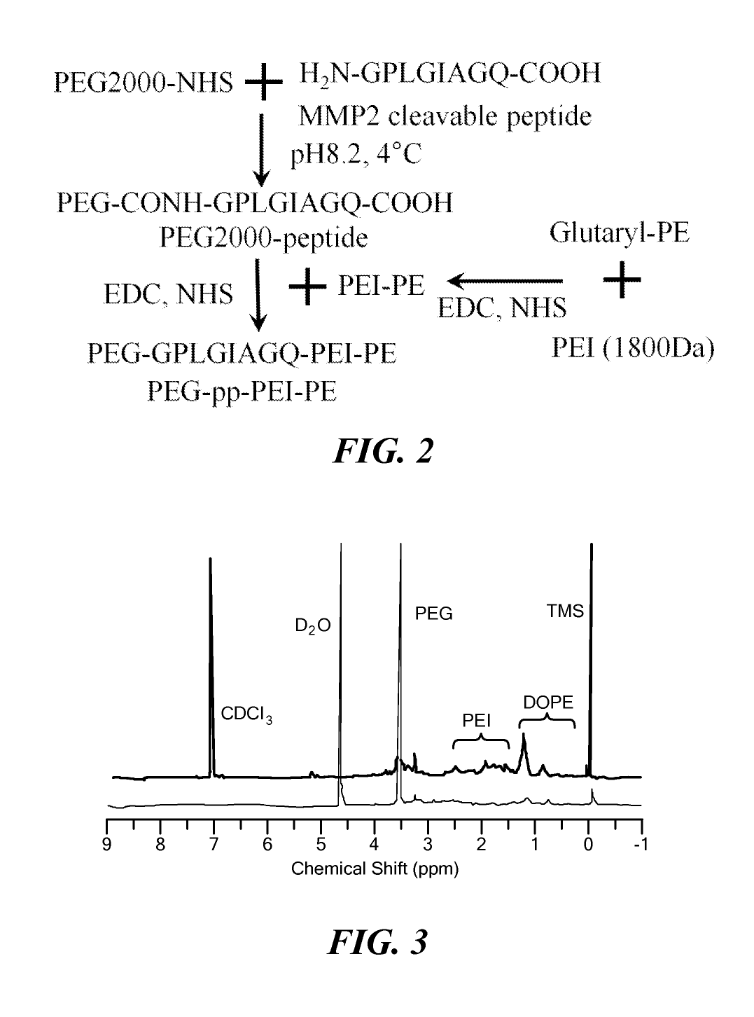

The three-step synthesis of PEG-pp-PEI-PE is shown in the FIG. 2. In previous work, PEG2000-peptide [13] and PEI-PE [6,7] have been successfully synthesized. Here, the same methods were used. Then, PEG-pp was conjugated with PEI-PE in the presence of the coupling reagents (NHS/EDC). FIG. 3 shows the ¹H NMR spectra of PEG-pp-PEI-PE. In CDCl3, the characteristic peaks of PEG-pp-PEI-PE were displayed [DOPE (—CH2-), 0.6-1.8 ppm; PEI (—CH2CH2NH—), 1.8-3 ppm; PEG (—CH2CH2O—), 3.60-3.65 ppm]. However, most peaks of PE were disappeared or significantly lowered when D₂O was used as solvent. This could be due to the formation of “core-shell” nanostructure in which the hydrophobic PE (and adjacent PEI) was entrapped in its “core” and isolated by the hydrophilic PEG “shell” in water, whereas the polymer would be fully dissolved in chloroform. The similar phenomenon was observed in the ¹H NMR spectra of the intermediate PEI-PE (data not shown). The integration of the characteristic peaks indicated that the molar ratio between PEG, PEI and PE was about 1:1:1.

### Example 3

### Micelle Formation and MMP2 Sensitivity

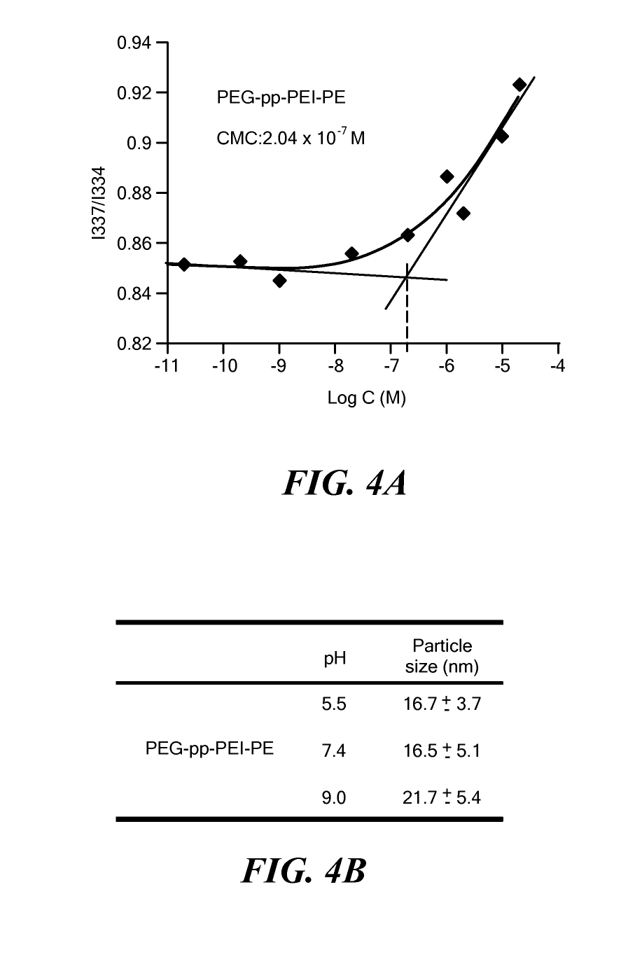

To confirm the micelle formation of PEG-pp-PEI-PE, the critical micelle concentration (CMC) (FIG. 4A) and particle size (FIG. 4B) were measured. The CMC of PEG-pp-PEI-PE was about 2.04×10⁻⁷ M, which is in the range of the CMC of the PEG-lipid micelles [15], indicating the formation of a micellar nanostructure. The PEG-pp-PEI-PE micelles were small and uniform and their particle size was consistent in a broad range of pH from 5.5 to 9.0, indicating the excellent stability of their micellar nanostructure.

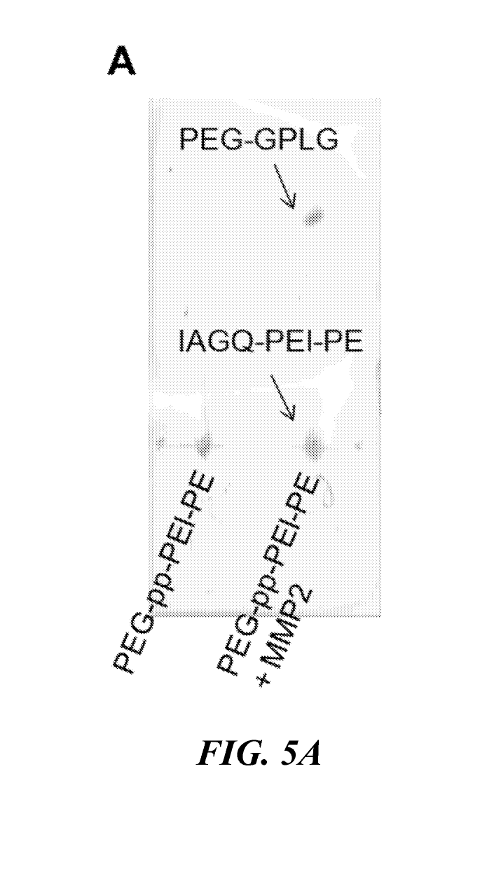

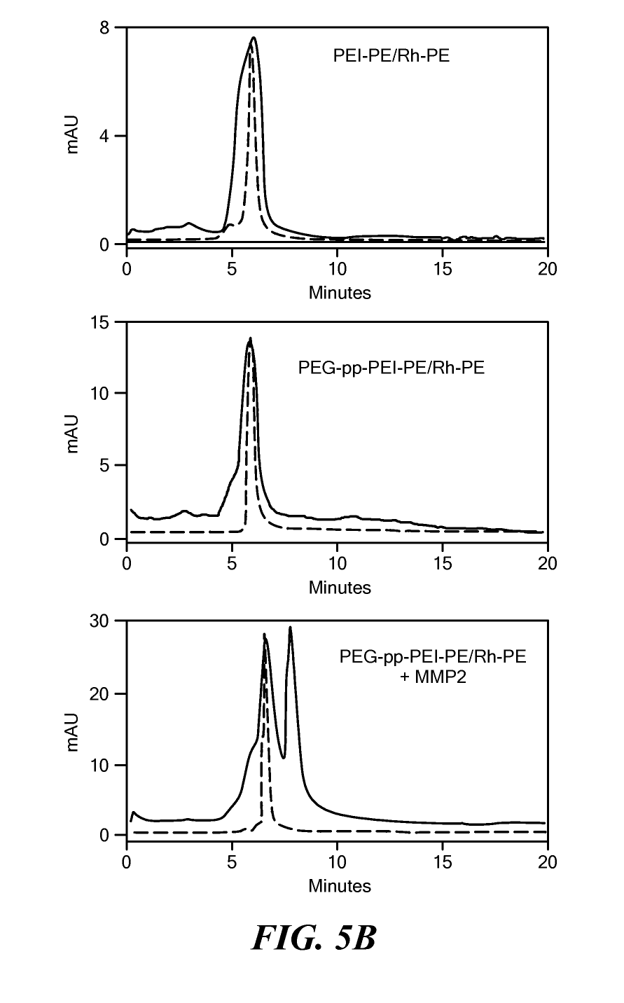

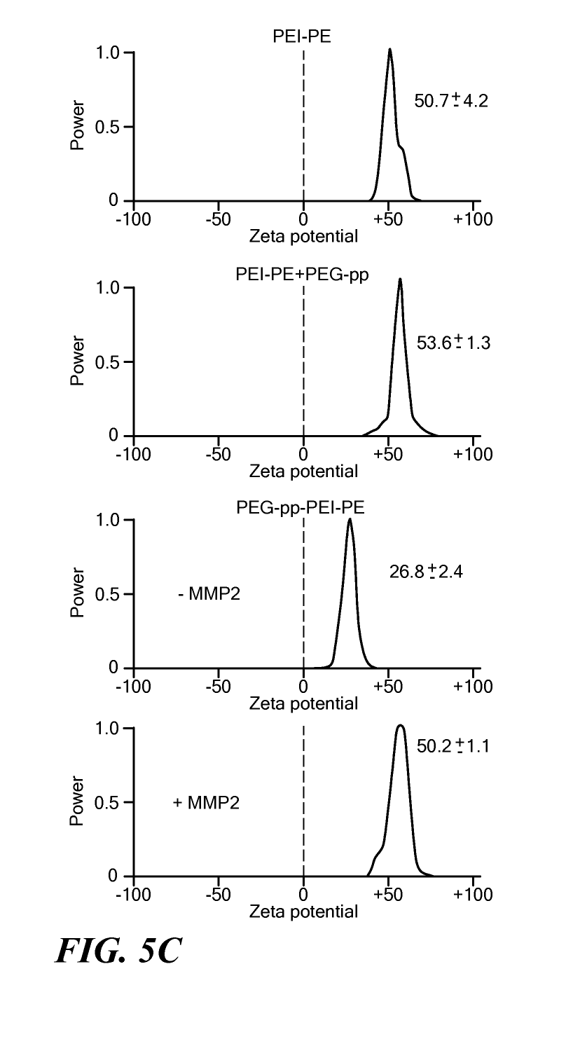

The MMP2 sensitivity of PEG-pp-PEI-PE was determined by enzymatic digestion followed by thin layer chromatography, size exclusion HPLC and zeta potential measurement. The MMP2 cleaved PEG-pp-PEI-PE at the site between glycine (G) and isoleucine (I) [12], resulting in two fractions. The released PEG moiety (PEG-GLPG) was visualized as a newspot on the TLC plate while the PEI-PE moiety (IAGQ-PEI-PE) could not move due to its high polarity (FIG. 5A). In the size exclusion chromatogram (FIG. 5B), the peaks of micelles formed by PEI-PE or PEG-pp-PEI-PE were indicated by fluorescent Rh-PE (red, discontinuous) due to the strong binding force between the Rh-PE and hydrophobic core of the micelles. After MMP2 treatment, the peak of PEG-GLPG was shown with a longer retention time but without fluorescence signal, while the IAGQ-PEI-PE was still form the micellar nanostructure as evidenced by the overlay between the UV and fluorescence signal. The data indicated that the PEG was released from the micelles and the micellar nanostructure was still remained after MMP2 cleavage. The stable micellar nanostructure ensures the high hydrophobic drug loading and low drug leakage before and after MMP2 cleavage in the in vitro and in vivo conditions.

As expected, conjugation of PEG-pp to PEI-PE significantly decreased the zeta potential of the formed conjugate from 50.7 4.2 to 26.8 2.4 mV, while no decrease in the zeta potential was observed by mixing of PEI-PE with PEG-pp (53.6 1.3 mV), supporting that only covalent conjugation between PEG-pp and PEI-PE could shield the positive charge of PEI. In contrast, the MMP2 cleavage removed the PEG corona from the micelles and exposed PEI resulting in an increase in the zeta potential (50.2 1.1 mV) (FIG. 5C).

### Example 4

### Preparation and Characterization of siRNA and PTX Loaded Micelles

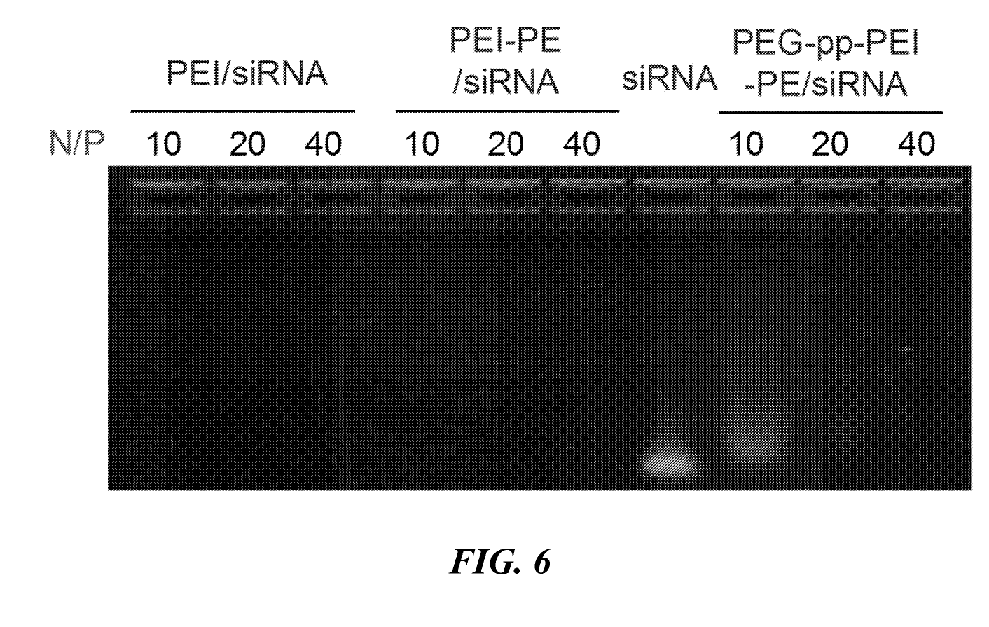

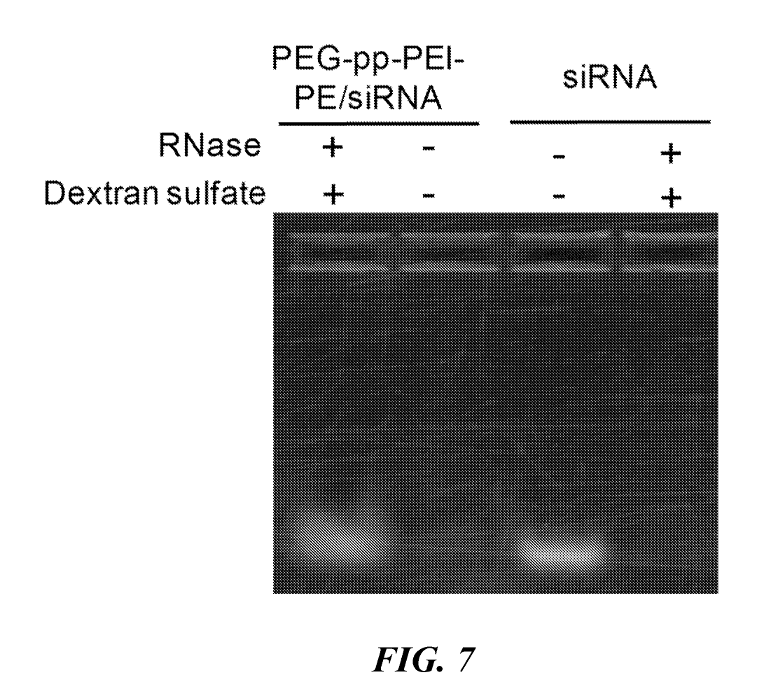

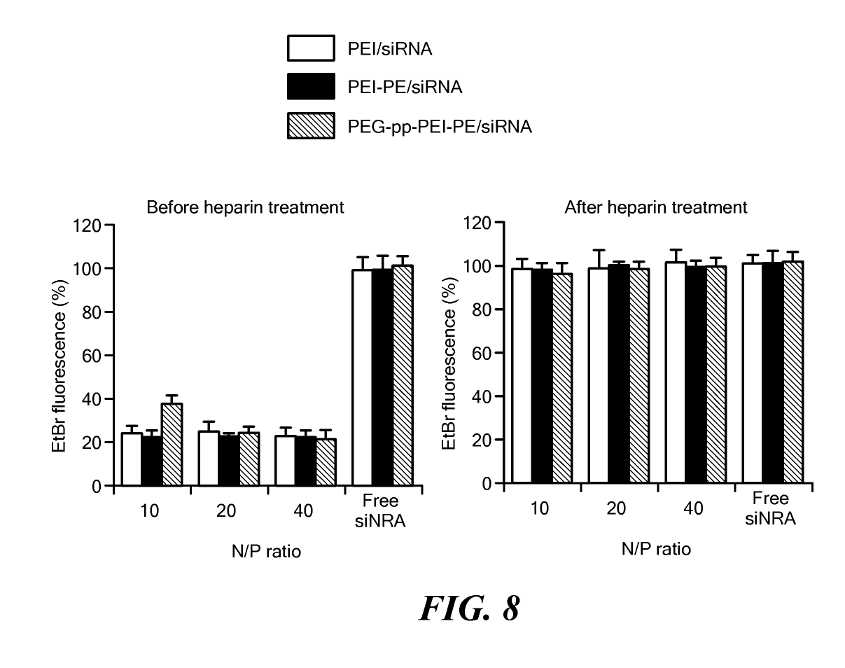

Free siRNA could be completely condensed by PEG-pp-PEI-PE at a nitrogen to phosphate ratio (N/P) of 40 (FIG. 6) and be protected thereafter from RNase degradation (FIG. 7). The condensed siRNA, however, could be dissociated from the siRNA complexes by negatively charged heparin, ensuring the efficient siRNA release upon cell entry (FIG. 8).

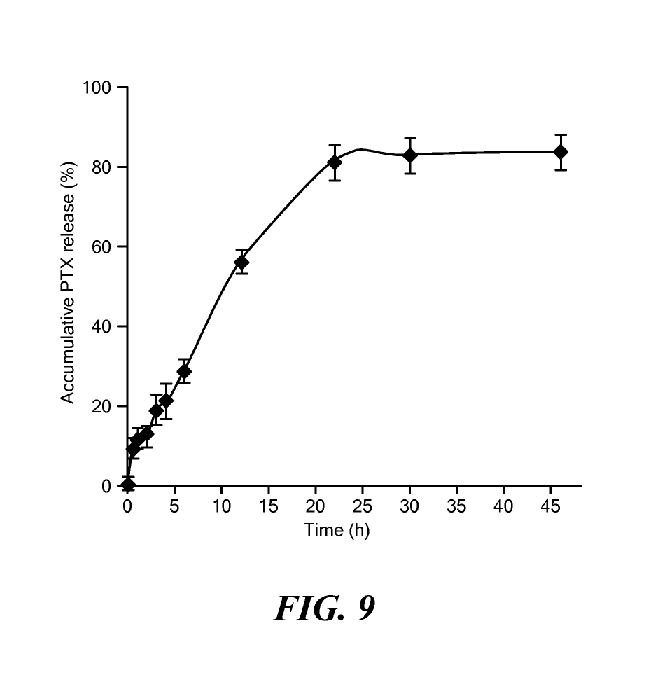

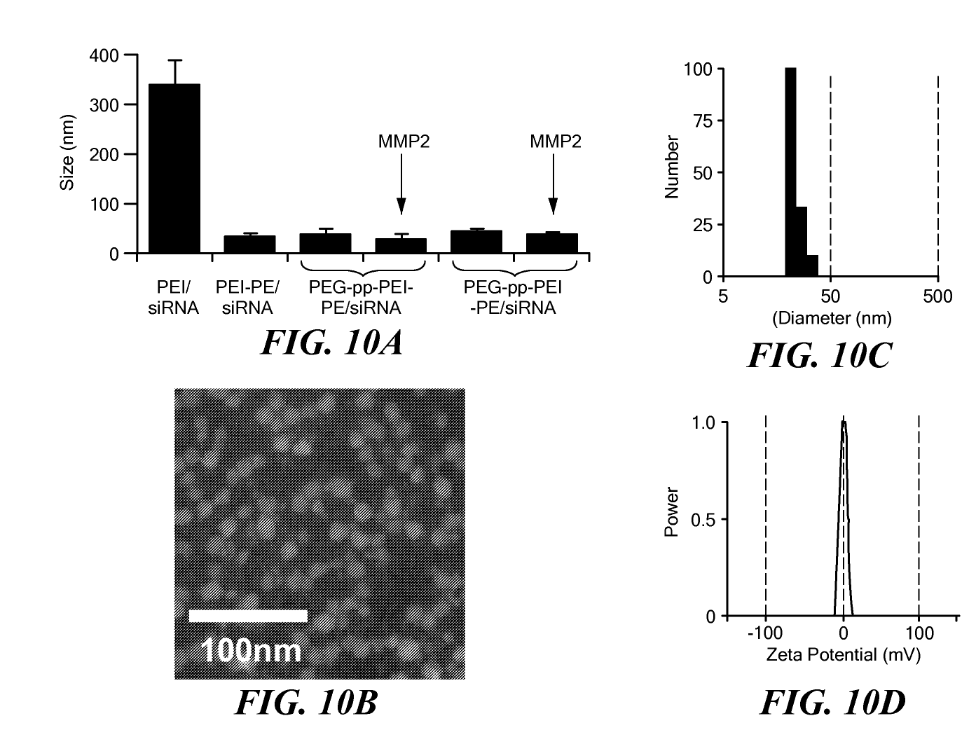

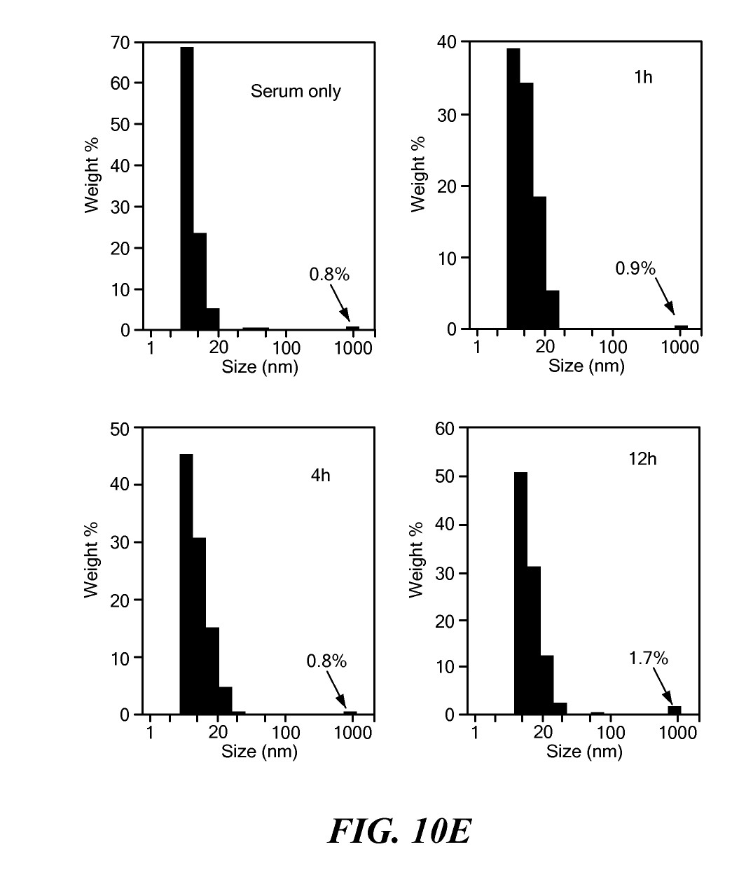

The poorly water-soluble PTX was loaded into the lipid core of the micelles via the hydrophobic interaction. The final drug loading was about 2.3 wt %, which was in agreement with the previous reports [15]. In the “sink condition”, only about 20% of the loaded PTX was released from PEG-pp-PEI-PE/PTX/siRNA complexes after 4 h incubation, while more than 80% drug was released after 20 h incubation (FIG. 9). This appropriate drug release profile ensured the efficient cell internalization of the loaded PTX as well as the sufficient dose of the released PTX for effective anticancer activity after endocytosis. The particle size of PEG-pp-PEI-PE/PTX/siRNA complexes was about 43 nm and wasn't increased much compared to that of PEG-pp-PEI-PE/siRNA complexes (about 37 nm) (FIG. 10A). They were much smaller than PEI/siRNA complexes (about 340 nm), probably due to their uniform “core-shell” nanostructure and less aggregation (FIGS. 10B and 10C). The zeta potential of PEG-pp-PEI-PE/PTX/siRNA nanoparticles was neutral (FIG. 10D), which is appropriate for in vivo nucleic acid delivery [5]. It was notable that the size of PEG-pp-PEI-PE/siRNA or PEG-pp-PEIPE/PTX/siRNA didn't change significantly before and after MMP2 cleavage and was similar to that of PEI-PE/siRNA, suggesting that PEG-pp-PEI-PE/PTX/siRNA complexes would be fairly stable during in vivo MMP2 cleavage in the tumor microenvironment (FIG. 10A).

To estimate the in vivo blood protein adsorption/interaction, PEG-pp-PEI-PE/PTX/siRNA nanoparticles were diluted by the mouse serum. In the presence of high content of serum, the fraction of large aggregates (1000 nm) caused by the interaction of PEG-pp-PEI-PE/PTX/siRNA and serum proteins was not significantly increased after 4 h incubation at 37° C. while just slightly increased from 0.8% to 1.7% after 12 h incubation (FIG. 10E). That's probably due to high density of PEG and appropriate PEG length on the surface of nanoparticles [13,16]. The PEG-pp-PEI-PE/PTX/siRNA nanoparticles with the minimized blood protein adsorption and small size are more likely to “escape” the capture by immune cells [16].

The sufficient drug loading, easy preparation procedure, small and uniform size, neutral charge, excellent stability, and negligible blood protein adsorption ensure the PEG-pp-PEI-PE micelles as an excellent platform for co-delivery of siRNA and hydrophobic drugs.

### Example 5

### In Vitro Cellular Uptake

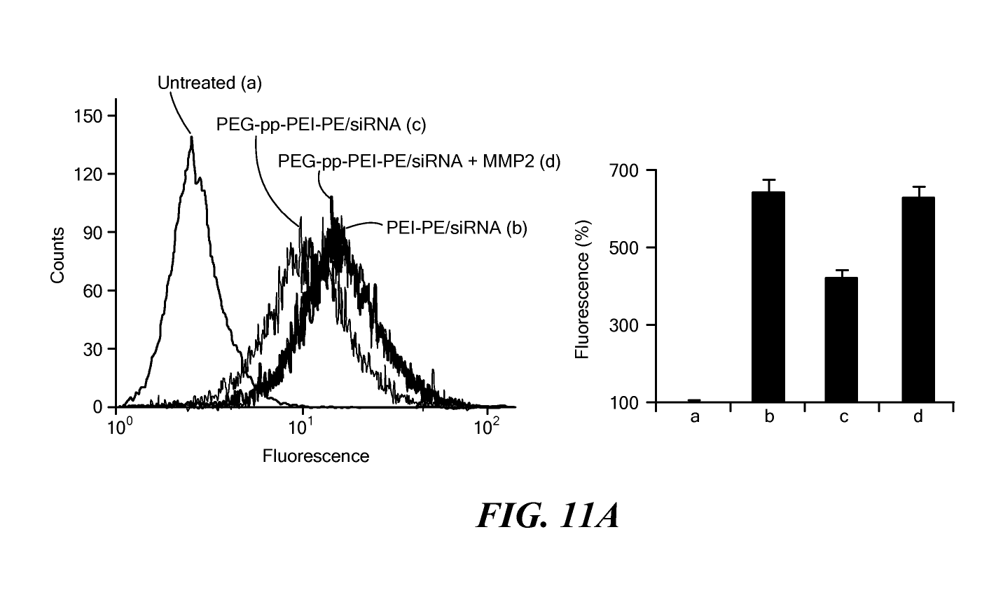

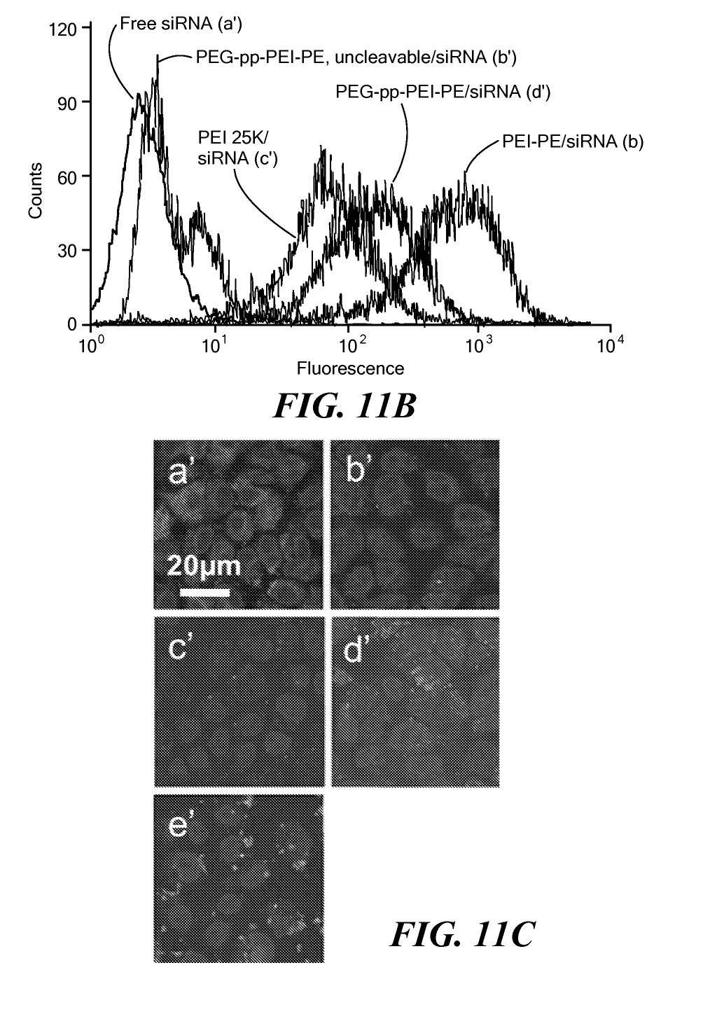

To study the influence of MMP2 on the cellular uptake of siRNA/polymer complexes, the PEG-pp-PEI-PE/siRNA complexes were pretreated with MMP2 before incubation with non-small cell lung cancer (NSCLC) cells (A549) in the serum-free medium. The cellular uptake of PEG-pp-PEI-PE/siRNA was significantly increased from 400% (c) to 650% (d) after MMP2 cleavage, the level similar to that of PEI-PE/siRNA (FIG. 11A), due to the PEG de-shielding and full exposure of PEI.

Without MMP2 pretreatment, PEG-pp-PEI-PE/siRNA showed higher transfection efficiency than that of the “gold standard” of transfection reagents, branched high molecular weight PEI (25 KDa), while its uncleavable counterpart didn't show significant transfection (FIG. 11B). The data indicated that the extracellular MMP2 in cancer cell media was sufficient to cleave the peptide linker [13] and the culture serum (protein) had little effect on the cell internalization of PEG-pp-PEI-PE/siRNA. However, their transfection efficiency was still lower than that of PEI-PE/siRNA, probably due to the strong interaction between the positively charged PEI-PE/siRNA and cell membrane. This is understandable. The N/P ratio of 40 used for preparation of PEI-PE/siRNA was much higher than the needed value (N/P<10) to siRNA (FIG. 6), resulting in “extra positive charge” on the PEI-PE/siRNA complexes, while the zeta potential of PEG-pp-PEI-PE/siRNA was around neutral. From an in vivo point of view, high positive charge may cause the nonspecific biodistribution and toxicity [5] and the near-neutral nanoparticles are preferred. The cellular uptake of the siRNA complexes was confirmed by confocal microscopy (FIG. 11C). Furthermore, the colocalization of siRNA (red, in web version) and the endosome/lysosome (green, in web version) indicated that the siRNA complexes most likely underwent endocytic pathway upon cell entry. The components (PEI and DOPE) of PEG-pp-PEI-PE were designed to facilitate the endosomal escape [5] and the following successful RNAi.

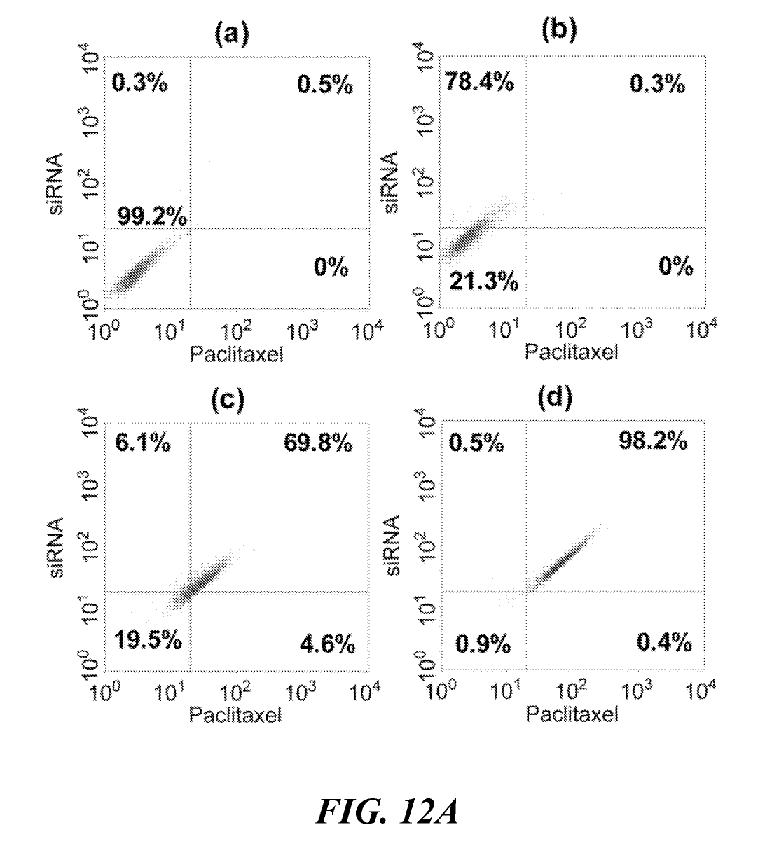

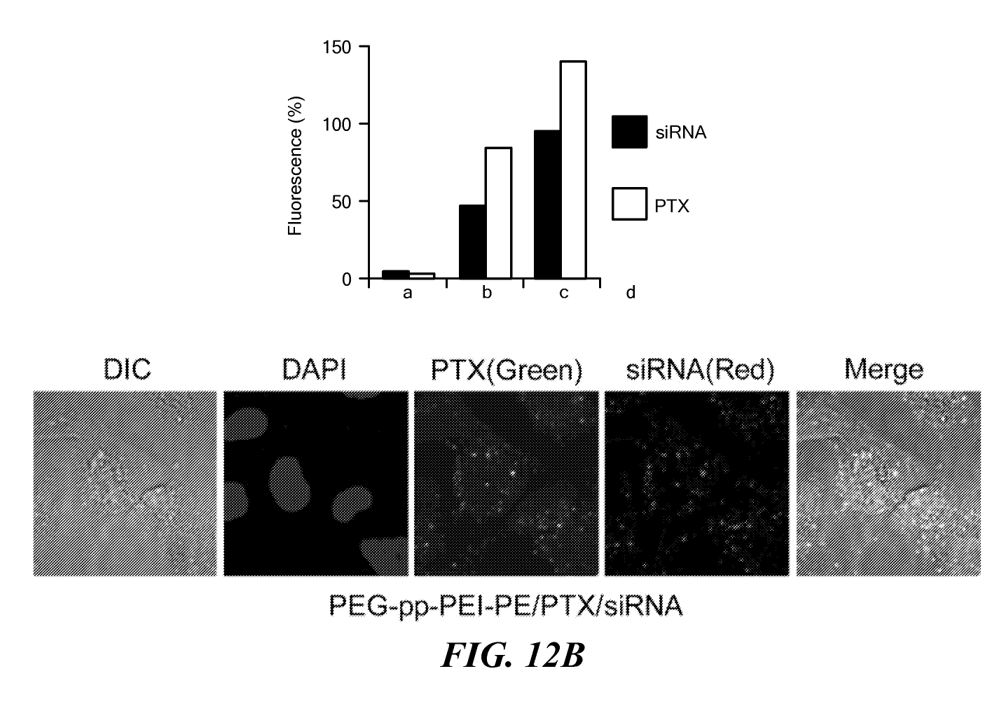

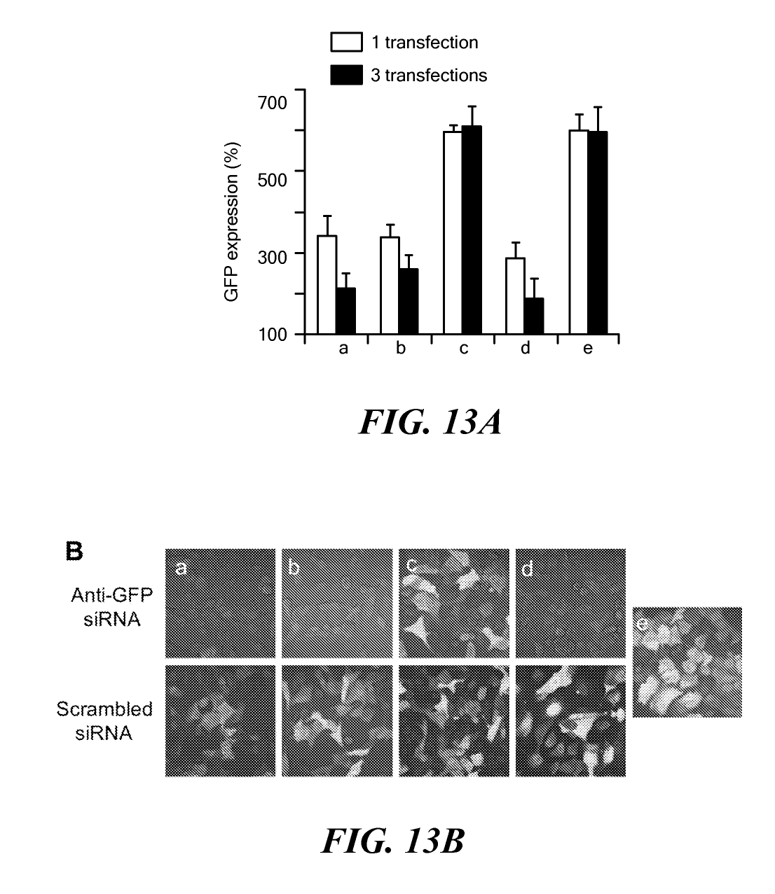

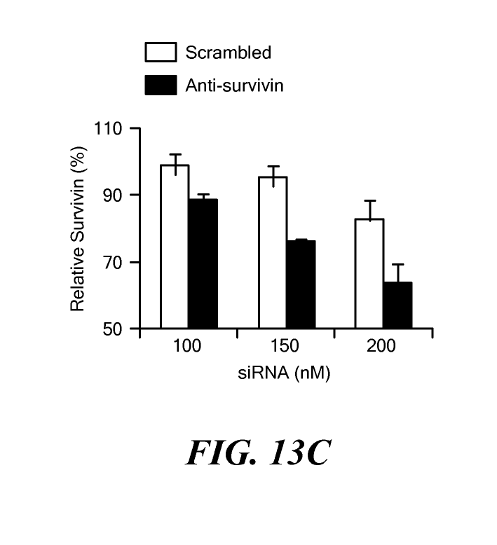

To estimate the in vitro co-delivery efficiency, the Oregon green-PTX and siGLO siRNA were co-loaded into PEG-pp-PEI-PE micelles. Compared to PEI25k which could only deliver siRNA but not PTX into cells, both MMP2 sensitive and nonsensitive PEG-pp-PEI-PE micelles were capable of co-delivery of siRNA and PTX. However, compared to the nonsensitive counterparts (69.8%), MMP2-sensitive micelles co-delivered siRNA and PTX to almost all cells (98.2%), a result of the MMP2-induced PEG de-shielding and PEI exposure (dot plot, FIG. 12A). The fluorescence intensity in the MMP2-sensitive micelle-treated cells was much higher than those in the nonsensitive micelle-treated ones (siRNA: 93.6% vs. 44.7%, PTX: 137.5% vs. 82.4%) (histogram, FIG. 13A). The co-delivery/colocalization of PTX and siRNA was further confirmed by the orange-yellow dots in the merged image under confocal microscopy (FIG. 12B).

### Example 6

### Gene Down-Regulation

To study the gene down-regulation of the anti-GFP siRNA, the GFP expressing (copGFP A549) cells were used as a cell model. In the presence of serum, one transfection of PEG-pp-PEI-PE/anti-GFP siRNA brought down the GFP expression to about 45% (b) of that of untreated cells (e), which was comparable to those of non-PEGylated siRNA complexes (a) and PEI25K/siRNA complexes. In contrast, the nonsensitive siRNA complexes (c) didn't show any GFP down-regulation. Three transfections led to more significant GFP down-regulation compared to one transfection (FIG. 13A). The data was confirmed by confocal microscopy as evidenced by the loss of the green fluorescence (FIG. 13B). It was notable that PEI25K induced high gene down-regulation although its cellular uptake efficiency was not higher than PEI-PE or PEG-pp-PEI-PE (FIG. 11B), probably due to its excellent buffering capacity-induced endosomal escape [5].

Besides, the therapeutic siRNA was used to evaluate the performance of PEG-pp-PEI-PE. Survivin, an inhibitor protein of apoptosis, is found up-regulated in malignant tumors, especially in drug resistant cells [17]. Anti-survivin siRNA have been used to down-regulate survivin and potentiate the anticancer activity of chemotherapeutics [18]. Here, an anti-survivin siRNA was complexed with PEG-pp-PEI-PE and transferred into PTX-resistant (A549 T24) NSCLC cells in the presence of serum. The surviving protein was down-regulated for about 30% at 150 nM siRNA and the down-regulation effect was dose-dependent (FIG. 13C). However, compared to the reporter gene, down-regulation of the therapeutic gene was relatively tough [19]. The similar gene down-regulation level by the survivin siRNA was observed in the previous study [20,21].

### Example 7

### In Vitro Synergistic Effect

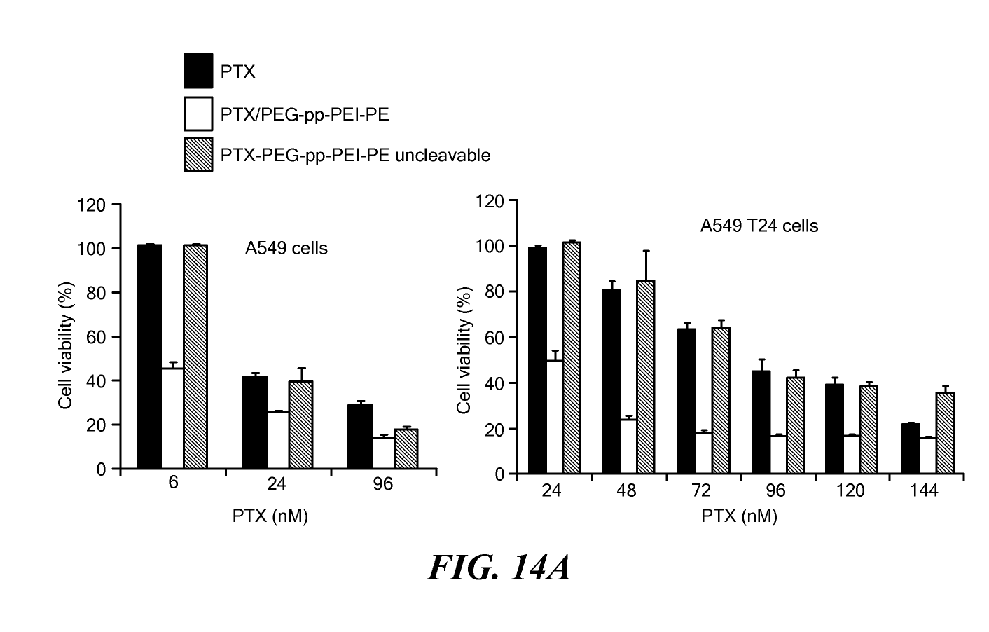

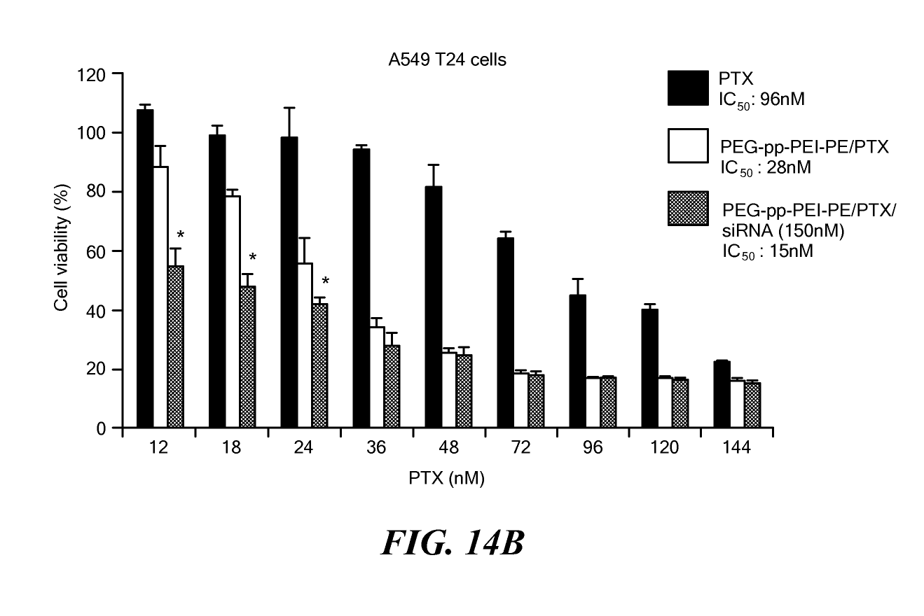

To study the synergistic effect of the anti-survivin siRNA and PTX co-loaded nanocarrier, both PTX-sensitive (A549) and -resistant (A549 T24) NSCLC cells were used. Compared to A549 cells with the IC50 of about 5.2 nM PTX, the A549 T24 cells were more resistant to PTX as evidenced by its high IC50 of about 96 nM PTX (data not shown). Incubation of PEG-pp-PEI-PE/PTX with A549 or A549 T24 cells significantly increased the cytotoxicity of PTX compared to those of free PTX or its nonsensitive micelles (FIG. 14A), probably due to the increased drug solubility and enhanced cellular uptake (FIG. 12A). Furthermore, the simultaneous delivery of anti-survivin siRNA and PTX significantly brought down the IC50 of PTX to about 15 nM (FIG. 14B). In contrast, the polymer by itself or in a complex with siRNA is very safe, and no cytotoxicity of antisurvivin siRNA at the used doses was observed (data not shown). Altogether, this enhanced antitumor activity was a result of the enhanced co-delivery efficiency and synergistic effect of PTX and anti-survivin siRNA [22].

### Example 8

### In Vivo Co-Delivery of siRNA and PTX

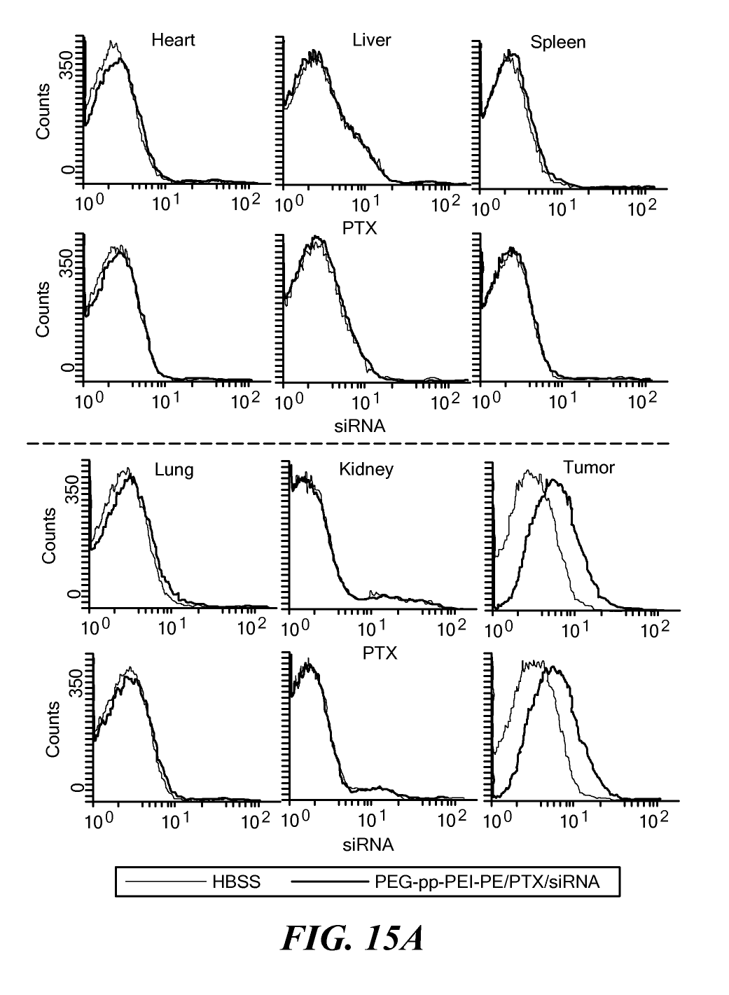

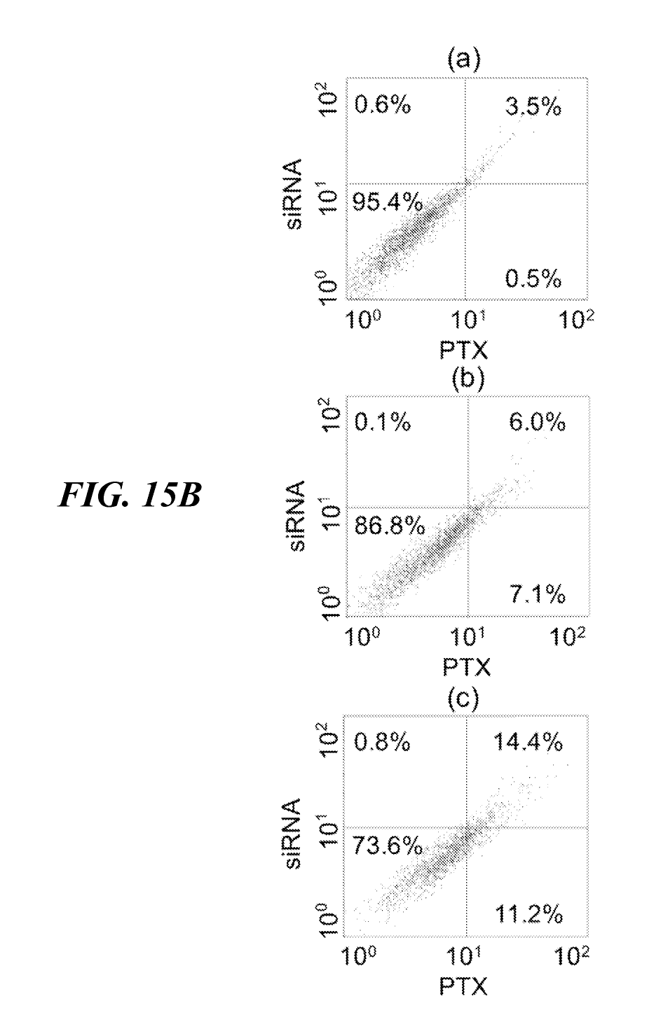

The in vivo co-delivery efficiency was studied on a NSCLC xenograft mouse model. Two hour after i.v. injection, siGLO siRNA and Oregon green PTX were predominately accumulated in tumor tissues and internalized by tumor cells, as evidenced by the high fluorescence of both siRNA and PTX in the tumor cells. In contrast, no obvious cell internalization of the fluorescent PTX or siRNA was observed in the major organs (FIG. 15A). The data indicated that PEG-pp-PEI-PE micelles were capable of targeted delivery of their payloads to the tumor via both the enhanced permeability and retention (EPR) effect and MMP2 sensitivity. The MMP2-mediated cleavage de-shielded PEG and exposed PEI, leading to the enhanced tumor cell internalization of the nanoparticles. In the tumor, about 14.4% of total cells internalized both siRNA and PTX after administration of PEG-pp-PEI-PE/PTX/siRNA. It was about 2.4-fold higher than that of its nonsensitive counterpart (6%) (FIG. 15B). The in vivo co-delivery efficiency was lower than the in vitro data (FIG. 12A), which was in agreement with previous studies [9]. Unlike the in vitro condition, the in vivo condition is more complicated and many factors including nonspecific tissue distribution [13], extracellular drug accumulation [13,23], and limited tissue penetration [24,25], influence the tumor cell internalization of drug and siRNA. Other factors such as low doses and non-optimized time of sampling also play an important role in the in vivo drug delivery. The optimization of dose regimen for in vivo drug delivery and antitumor efficacy study are undergoing.

As used herein, “consisting essentially of” does not exclude materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

### REFERENCES

1. Song E, Lee S K, Wang J, Ince N, Ouyang N, Min J, et al. RNA interference targeting Fas protects mice from fulminant hepatitis. Nat Med 2003; 9:347-51.
2. de Fougerolles A, Vornlocher H P, Maraganore J, Lieberman J. Interfering with disease: a progress report on siRNA-based therapeutics. Nat Rev Drug Discov 2007; 6:443-53.
3. Oh Y K, Park T G. siRNA delivery systems for cancer treatment. Adv Drug Deliv Rev 2009; 61:850-62.
4. De Paula D, Bentley M V, Mahato R I. Hydrophobization and bioconjugation for enhanced siRNA delivery and targeting. Rna 2007; 13:431-56.
5. Zhu L, Mahato R I. Lipid and polymeric carrier-mediated nucleic acid delivery. Expert Opin Drug Deliv 2010; 7:1209-26.
6. Sawant R R, Sriraman S K, Navarro G, Biswas S, Dalvi R A, Torchilin V P. Polyethyleneimine-lipid conjugate-based pH-sensitive micellar carrier for gene delivery. Biomaterials 2012; 33:3942-51.
7. Navarro G, Sawant R R, Biswas S, Essex S, Tros de Ilarduya C, Torchilin V P. P-glycoprotein silencing with siRNA delivered by DOPE-modified PEI overcomes doxorubicin resistance in breast cancer cells. Nanomedicine (Load) 2012; 7: 65-78.
8. Wang Y, Gao S, Ye W H, Yoon H S, Yang Y Y. Co-delivery of drugs and DNA from cationic core-shell nanoparticles self-assembled from a biodegradable copolymer. Nat Mater 2006; 5:791-6.
9. Sun T M, Du J Z, Yao Y D, Mao C Q, Dou S, Huang S Y, et al. Simultaneous delivery of siRNA and paclitaxel via a “two-in-one” micelleplex promotes synergistic tumor suppression. ACS Nano 2011; 5:1483-94.
10. Chen A M, Zhang M, Wei D, Stueber D, Taratula O, Minko T, et al. Co-delivery of doxorubicin and Bcl-2 siRNA by mesoporous silica nanoparticles enhances the efficacy of chemotherapy in multidrug-resistant cancer cells. Small 2009; 5: 2673-7.
11. Zhu L, Torchilin V P. Stimulus-responsive nanopreparations for tumor targeting. Integr Biol 2013; 5:96-107.
12. Zhu L, Kate P, Torchilin V P. Matrix metalloprotease 2-responsive multifunctional liposomal nanocarrier for enhanced tumor targeting. ACS Nano 2012; 6: 3491-8.
13. Zhu L, Wang T, Perche F, Taigind A, Torchilin V P. Enhanced anticancer activity of nanopreparation containing an MMP2-sensitive PEG-drug conjugate and cell-penetrating moiety. Proc Natl Acad Sci USA 2013; 110:17047-52.
14. Kortylewski M, Kujawski M, Wang T, Wei S, Zhang S, Pilon-Thomas S, et al. Inhibiting Stat3 signaling in the hematopoietic system elicits multicomponent antitumor immunity. Nat Med 2005; 11:1314-21.
15. Lukyanov A N, Torchilin V P. Micelles from lipid derivatives of water-soluble polymers as delivery systems for poorly soluble drugs. Adv Drug Deliv Rev 2004; 56:1273-89.
16. Walkey C D, Olsen J B, Guo H, Emili A, Chan W C. Nanoparticle size and surface chemistry determine serum protein adsorption and macrophage uptake. J Am Chem Soc 2012; 134:2139-47.
17. Ambrosini G, Adida C, Altieri D C. A novel anti-apoptosis gene, survivin, expressed in cancer and lymphoma. Nat Med 1997; 3:917-21.
18. Altieri D C. Survivin, versatile modulation of cell division and apoptosis in cancer. Oncogene 2003; 22:8581-9.
19. Zhu L, Mahato R I. Targeted delivery of siRNA to hepatocytes and hepatic stellate cells by bioconjugation. Bioconjug Chem 2010; 21:2119-27.
20. Li S-D, Huang L. Targeted delivery of antisense oligodeoxynucleotide and small interference RNA into lung cancer cells. Mol Pharmacol 2006; 3:579-88.
21. Salzano G, Riehle R, Navarro G, Perche F, De Rosa G, Torchilin V P. Polymeric micelles containing reversibly phospholipid-modified anti-survivin siRNA: a promising strategy to overcome drug resistance in cancer. Cancer Lett 2014; 343:224-31.
22. Shen J, Yin Q, Chen L, Zhang Z, Li Y. Co-delivery of paclitaxel and surviving shRNA by pluronic P85-PEI/TPGS complex nanoparticles to overcome drug resistance in lung cancer. Biomaterials 2012; 33:8613-24.
23. Kirpotin D B, Drummond D C, Shao Y, Shalaby M R, Hong K, Nielsen U B, et al. Antibody targeting of long-circulating lipidic nanoparticles does not increase tumor localization but does increase internalization in animal models. Cancer Res 2006; 66:6732-40.
24. Zorko M, Langel U. Cell-penetrating peptides: mechanism and kinetics of cargo delivery. Adv Drug Deliv Rev 2005; 57:529-45.
25. Cabral H, Matsumoto Y, Mizuno K, Chen Q, Murakami M, Kimura M, et al. Accumulation of sub-100 nm polymeric micelles in poorly permeable tumours depends on size. Nat Nanotechnol 2011; 6:815-23.
26. Benjamin E. Turk, Lisa L. Huang, Elizabeth T. Piro, and Lewis C. Cantley. Determination of protease cleavage site motifs using mixture-based oriented peptide libraries. Nat Biotechnol 2001; 19:661-667.
27. Steven J. Kridel, Emily Chen, Lakshmi P Kotra, Eric W. Howard, Shahriar Mobashery, and Jeffrey W. Smith. Substrate Hydrolysis by Matrix Metalloproteinase-9. J. Biol. Chem. 2001; 276:20572-20578.

What is claimed is:

1. A protease-sensitive, polynucleotide-binding molecule comprising:
(1) an uncharged hydrophilic polymer;
(2) a peptide having a target cleavage site for a protease, wherein the peptide is attached to the uncharged hydrophilic polymer by a first covalent linkage;
(3) a positively-charged polymer, wherein the positively-charged polymer is attached to the peptide by a second covalent linkage, and wherein the positively-charged polymer binds one or more polynucleotide molecules; and
(4) a phospholipid, wherein the phospholipid is attached to the positively-charged polymer by a third covalent linkage;
wherein the uncharged hydrophilic polymer, the peptide, the positively-charged polymer, and the phospholipid are present in about a 1:1:1:1 molar ratio.

2. The molecule of claim 1, wherein the uncharged polymer is selected from the group consisting of polyethylene glycol, polyvinylpyrrolidone, and polyacrylamide.

3. The molecule of claim 2, wherein the uncharged polymer is polyethylele glycol

4. The molecule of claim 3, wherein the polyethlylene glycol has an average molecular weight from about 1000 to about 5000 daltons.

5. The molecule of claim 4, wherein the polyethylene glycol has an average molecular weight of about 2000 daltons.

6. The molecule of claim 1, wherein the target cleavage site is specific for a matrix metalloproteinase.

7. The molecule of claim 6, wherein the peptide comprises the amino acid sequence Gly-Pro-Leu-Gly-Ile-Ala-Gly-Gln (SEQ ID NO:1).

8. The molecule of claim 6, wherein the target cleavage site is a matrix metalloproteinase cleavage site from a protein selected from the group consisting of Aggrecan, Big endothelin-1, Brevican/BEHAB, Collagen-α1 (I), Collagen-α1(X), Decorin, FGFR-1, Galectin-3, IGFBP-3, IL-1β, Laminin-5 γ2-chain, α2-Macroglobulin, MCP-3, Pregnancy zone protein, Pro-MMP-1, Pro-MMP-2, SPARC, Substance P, Betaglycan, Dentin, Integrin-αV, Integrin-α6, Integrin-αX, Integrin-α9, NG2 proteoglycan, Neurocan, and PAI-3.

9. The molecule of claim 1, wherein the peptide comprises the sequence Xaa₁-Xaa₂-Xaa₃-Xaa₄-Xaa₅-Xaa₆, wherein:
Xaa₁ is selected from the group consisting of Ala, Ile, Pro, and Val;
Xaa₂ is any amino acid;
Xaa₃ is selected from the group consisting of Ala, Asn, Gln, Glu, Gly, Ser, and Thr;
Xaa₄ is selected from the group consisting of Arg, Ile, Leu, Met, Phe, and Tyr;
Xaa₅ is any amino acid; and
Xaa₆ is selected from the group consisting of Ala, Gln, Gly, Met, Ser, Tyr, and Val;
and wherein the protease cleaves the peptide bond between Xaa₃ and Xaa₄ (SEQ ID NO: 2).

10. The molecule of claim 9, wherein:
Xaa₂ is selected from the group consisting of Ala, Arg, Asn, Glu, Gly, Leu, Met, Phe, Tyr, and Val; and
Xaa₅ is selected from the group consisting of Ala, Arg, Asn, Ile, Leu, Lys, Met, Ser, Thr, Tyr, and Val (SEQ ID NO:3).

11. The molecule of claim 1, wherein the positively-charged polymer is selected from the group consisting of polyethylenimine, polylysine, a cationic peptide, poly(dl-lactide-co-glycolide), poly(amidoamine), and poly(propylenimine).

12. The molecule of claim 11, wherein the positively-charged polymer is polyethylenimine

13. The molecule of claim 12, wherein the polyethylenimine has a molecular weight from about 500 daltons to about 5000 daltons.

14. The molecule of claim 13, wherein the polyethylenimine has a molecular weight of about 1800 daltons.

15. The molecule of claim 12, wherein the polyethylenimine has a branched structure.

16. The molecule of claim 1, wherein the phospholipid is selected from the group consisting of phosphatidic acid, phosphatidylethanolamine, phosphatidylcholine, phosphatidylserine, phosphatidylinositol, phosphatidylglycerol, and a sphingolipid.

17. The molecule of claim 16, wherein the phospholipid comprises fatty acid side chains each having from 12-20 carbon atoms.

18. The molecule of claim 17, wherein the fatty acid side chains are saturated, monounsaturated, diunsaturated, or triunsaturated.

19. The molecule of claim 18, wherein the phospholipid is phosphtatidylethanolamine.

20. The molecule of claim 19, wherein the phosphatidylethanolamine is 1,2-dioleoyl-sn-glycero-3-phosphoethanolamine.

21. The molecule of claim 1, wherein each of the first, second, and third covalent linkages is selected from a group consisting of a peptide bond, amide bond, ester bond, ether bond, alkyl bond, carbonyl bond, alkenyl bond, thioether bond, disulfide bond, and azide bond.

22. The molecule of claim 1, wherein the first, second, and third covalent linkages are peptide bonds.

23. A nanoparticle composition for delivery of a polynucleotide to a cell or tissue that overexpresses a protease, the composition comprising a plurality of molecules of claim 1 suspended in an aqueous medium and aggregated to form one or more nanoparticles.

24. The nanoparticle composition of claim 23, further comprising one or more polynucleotides non-covalently bound to the positively-charged polymers of said molecules.

25. The nanoparticle composition of claim 24, wherein the one or more polynucleotides are selected from the group consisting of a single-stranded RNA, a double-stranded RNA, and single-stranded DNA, and a double-stranded RNA.

26. The nanoparticle composition of claim 25, wherein the one or more polynucleotides are siRNA.

27. The nanoparticle of composition of claim 26, wherein the composition has two or more polynucleotides, and wherein the polynucleotides are two or more different species of siRNA.

28. The nanoparticle composition of claim 24, wherein the polynucleotide is an antisense oligonucleotide.

29. The nanoparticle composition of claim 24, wherein the polynucleotide is an siRNA or antisense oligonucleotide suitable for treating cancer.

30. The nanoparticle composition of claim 24, wherein the polynucleotide targets the expression of one or more genes selected from the group consisting of survivin, Eg5, EGFR, XIAP, CDC45L, SUV420h1, WEE1, HDAC2, RBX 1, CDK4, CSN5, FOXM1, R1 (RAM2), LSD1, CSTF2, Nectin-4, ERCC6L, PKIB, NAALADL2, PRMT1, COPZ1, SYNGR4, P-glycoprotein, VEGFR, and VEGF.

31. The nanoparticle composition of claim 24, wherein the nanoparticle composition has a nitrogen:phosphate ratio from about 1:5 to about 1:50.

32. The nanoparticle composition of claim 23, wherein the nanoparticles are micelles.

33. The nanoparticle composition of claim 32, wherein the micelles have an average diameter from about 10 nm to about 50 nm.

34. The nanoparticle composition of claim 23, wherein the cell or tissue that overexpresses a protease is associated with cancer.

35. The nanoparticle composition of claim 34, wherein the cancer is selected from the group consisting of ovarian cancer, breast cancer, prostate cancer, uterine cancer, cervical cancer, prostate cancer, and melanoma, pancreatic cancer, tongue cancer, bladder cancer, carcinoma, gastric cancer, stomach cancer, liver cancer, hepatoma, colorectal cancer, lung cancer, gall bladder cancer, nasopharyngeal cancer, oral cancer, squamous cell cancer, kidney cancer, renal cancer, laryngeal cancer, leukemia, bone cancer, skin cancer, basal cell carcinoma, extra-gastrointestinal stromal cancer, and thyroid cancer.

36. The nanoparticle composition of claim 23, wherein the peptide of said molecules is cleavable by a protease.

37. The nanoparticle composition of 36, wherein cleavage of the peptide causes release of the uncharged hydrophilic polymers from the nanoparticles.

38. The nanoparticle composition of claim 24, wherein the peptide of said molecules is cleavable by a protease, and said cleavage results in increased cellular uptake of bound polynucleotides.

39. The nanoparticle composition of claim 23, further comprising a hydrophobic pharmaceutical agent.

40. The nanoparticle composition of claim 39, wherein the pharmaceutical agent is an anti-cancer agent.

41. The nanoparticle composition of claim 40, wherein the pharmaceutical agent is selected from the group consisting of altretamine, aminoglutethimide, amsacrine (m-AMSA), azacitidine, baccatin III, bleomycin, busulfan, carmustine (BCNU), chlorambucil, cytarabine HCl, dacarbazine, dactinomycin, daunorubicin, docetaxel, doxorubicin, etoposide (VP-16), 5-fluorouracil, floxuridine, flutamide, hydroxyurea, ifosfamide, leuprolide acetate, lomustine (CCNU), melphalan, methotrexate, mitomycin, mitotane (o.p′-DDD), octreotide, paclitaxel, pentostatin, plicamycin, procarbazine HCl, semustine (methyl-CCNU), streptozocin, tamoxifen citrate, teniposide (VM-26), thioguanine, thiotepa, vindesine, vinblastine, and vincristine sulfate.

42. The nanoparticle composition of claim 41, wherein the pharmaceutical agent is paclitaxel.

43. The nanoparticle composition of claim 23, wherein the protease is a matrix metalloproteinase.

44. The nanoparticle composition of claim 43, wherein the matrix metalloproteinase is MMP-2 or MMP-9.

45. The nanoparticle composition of claim 23, wherein the composition consists of a plurality of said molecules.

46. A pharmaceutical composition comprising the nanoparticle composition of claim 23 suspended in an aqueous buffer.

47. A pharmaceutical composition comprising the nanoparticle composition of claim 24 suspended in an aqueous buffer.

48. A pharmaceutical composition comprising the nanoparticle composition of claim 39 suspended in an aqueous buffer.

49. The pharmaceutical composition of any one of claims 46 to 48, further comprising an excipient.

50. A method of making the protease-sensitive, polynucleotide-binding molecule of claim 1, the uncharged polymer having a first reactive group, the peptide having a target cleavage site for a protease and having second and third reactive groups, the positively-charged polymer having fourth and fifth reactive groups, and the phospholipid having a sixth reactive group, the method comprising the steps of:
(1) reacting the first reactive group on the uncharged hydrophilic polymer with the second reactive group on the peptide, wherein the uncharged hydrophilic polymer and the peptide are present in about a 1:1 molar ratio, to create the first covalent linkage;
(2) reacting the third reactive group on the peptide with the fourth reactive group on the positively-charged polymer, wherein the peptide and the positively-charged polymer are present in about a 1:1 molar ratio, to create the second covalent linkage; and
(3) reacting the fifth reactive group on the positively-charged polymer with the sixth reactive group on the phospholipid, wherein the positively-charged polymer and the phospholipid are present in about a 1:1 molar ratio, to create the third covalent linkage.

51. The method of claim 50, wherein the steps are performed in the following order: (1), (2), and (3).

52. The method of claim 50, wherein the steps are performed in the following order: (1), (3), and (2).

53. The method of claim 50, wherein the steps are performed in the following order: (2), (1), and (3).

54. The method of claim 50, wherein the steps are performed in the following order: (2), (3), and (1).

55. The method of claim 50, wherein the steps are performed in the following order: (3), (1), and (2).

56. The method of claim 50, wherein the steps are performed in the following order: (3), (2), and (1).

57. The method of any one of claims 50 to 56, wherein the hydrophilic polymer is polyethylene glycol.

58. The method of claim 57, wherein the polyethlylene glycol has an average molecular weight from about 1000 to about 5000 daltons.

59. The method of claim 58, wherein the polyethylene glycol is polyethylene glycol 2000-N-hydroxysuccinamide ester.

60. The method of any one of claims 50 to 56, wherein the positively-charged polymer is polyethylenimine.

61. The method of claim 60, wherein the polyethylenimine has a molecular weight from about 500 daltons to about 5000 daltons.

62. The method of claim 61, wherein the polyethylenimine has an average molecular weight of about 1800 daltons.

63. The method of any one of claims 50 to 56, wherein the phospholipid is phosphtatidylethanolamine.

64. The method of claim 63, wherein the phosphatidylethanolamine is 1,2-dioleoyl-sn-glycero-3-phosphoethanolamine-N-(glutaryl).

65. The method of any one of claims 50 to 56, wherein the peptide comprises Gly-Pro-Leu-Gly-Ile-Ala-Gly-Gln (SEQ ID NO:1).

66. The method of any one of claims 50 to 56, wherein each of the first, second, and third covalent linkages is independently selected from the group consisting of a peptide bond, amide bond, ester bond, ether bond, alkyl bond, carbonyl bond, alkenyl bond, thioether bond, disulfide bond, and azide bond.

67. The method of claim 66, wherein the first, second, and third covalent linkages are peptide bonds.

68. The method of any one of claims 52 and 55, wherein the hydrophilic polymer is polyethylene glycol 2000-N-hydroxysuccinamide ester, the peptide comprises Gly-Pro-Leu-Gly-Ile-Ala-Gly-Gln (SEQ ID NO:1), the positively-charged polymer is branched polyethylenimine having an average molecular weight of about 1800 daltons, and the phospholipid is 1,2-dioleoyl-sn-glycero-3-phosphoethanolamine-N-(glutaryl).

69. The method of claim 68, wherein:
step (1) comprises:
(a1) reacting the peptide and polyethylene glycol 2000-N-hydroxysuccinimide ester in a 1.2:1 molar ratio in an aqueous solution to create a peptide-polyethlyne glycol product; and
(b1) removing the unreacted peptide; and
step (2) comprises:
(a2) reacting the peptide-polyethylene glycol product from step (1)(a) with a 20-fold molar excess of N-(3-dimethylaminopropyl)N′-ethylcarbodiimide hydrochloride and N-hydroxysuccinimide to create activated peptide-polyethylene glycol product;
(b2) reacting the activated peptide-polyethylene glycol product from step (2)(a) with the polyethylenimine-phosphoethanolamine product from step (3)(b) in a 1:1 molar ratio in the presence of a trace amount of triethylamine to create said protease-sensitive, polynucleotide-binding molecule; and
(c2) dialyzing the product of the reaction in (b) against H₂O; and
step (3) comprises:
(a3) reacting 1,2-dioleoyl-sn-glycero-3-phosphoethanolamine-N-(glutaryl) with a 20-fold molar excess of N-(3-dimethylaminopropyl)N′-ethylcarbodiimide hydrochloride and N-hydroxysuccinimide to create activated 1,2-dioleoyl-sn-glycero-3-phosphoethanolamine-N-(glutaryl);
(b3) reacting the activated 1,2-dioleoyl-sn-glycero-3-phosphoethanolamine-N-(glutaryl) product of (a) with branched polyethylenimine having an average molecular weight of about 1800 daltons at a 1:1 molar ratio in the presence of a trace amount of triethylamine at room temperature to create a polyethylenimine-phosphoethanolamine product; and
(c3) dialyzing the reaction against H₂O.

70. A method of making a nanoparticle composition comprising the protease-sensitive, polynucleotide-binding molecule of claim 1, the method comprising the steps of:
(1) providing a solution of said molecule in a non-aqueous solvent; and
(2) replacing the non-aqueous solvent with an aqueous medium to form an aqueous suspension comprising nanoparticles, the nanoparticles comprising aggregates of a plurality of the protease-sensitive, polynucleotide-binding molecules.

71. The method of claim 70, wherein step (2) comprises dialyzing the solution of protease-sensitive, polynucleotide-binding molecule against an aqueous medium to form the nanoparticles.

72. The method of claim 70, wherein step (2) comprises:
(a) evaporating the non-aqueous solvent to form a dry film of the protease-sensitive, polynucleotide-binding molecule; and
(b) suspending the dry film in an aqueous medium to form the nanoparticles.

73. The method of claim 70, wherein the nanoparticle composition consists of a plurality of the protease-sensitive, polynucleotide-binding molecules.

74. The method of claim 70, further comprising the step of adding a hydrophobic pharmaceutical agent to the solution of protease-sensitive, polynucleotide-binding molecule in a non-aqueous solvent, wherein the nanoparticles produced by replacing the non-aqueous solvent with an aqueous medium comprise the hydrophobic pharmaceutical agent.

75. The method of claim 70, further comprising the step of adding a hydrophobic pharmaceutical agent to the aqueous suspension comprising nanoparticles, whereby the hydrophobic pharmaceutical agent is incorporated into the nanoparticles.

76. The method of any one of claims 70 to 75, further comprising the step of adding one or more polynucleotides to the aqueous suspension comprising nanoparticles, whereby the one or more polynucleotides become non-covalently bound to the positively-charged polymers of said nanoparticles.

77. A method of treating in a subject a disease or condition associated with expression of a protease, the method comprising administering the nanoparticle composition of claim 23 to a subject having or suspected of having the disease or condition.

78. The method of claim 77, wherein the disease or condition is cancer.

79. The method of claim 78, wherein the cancer is selected from the group consisting of ovarian cancer, breast cancer, prostate cancer, uterine cancer, cervical cancer, prostate cancer, and melanoma, pancreatic cancer, tongue cancer, bladder cancer, carcinoma, gastric cancer, stomach cancer, liver cancer, hepatoma, colorectal cancer, lung cancer, gall bladder cancer, nasopharyngeal cancer, oral cancer, squamous cell cancer, kidney cancer, renal cancer, laryngeal cancer, leukemia, bone cancer, skin cancer, basal cell carcinoma, extra-gastrointestinal stromal cancer, and thyroid cancer.

80. The method of claim 77, wherein the nanoparticle composition is administered by a parenteral route.

81. The method of claim 80, wherein the parenteral administration route is selected from the group consisting of intravascular administration, peri- and intra-tissue administration, subcutaneous injection or deposition, subcutaneous infusion, intraocular administration, and direct application at or near the site of neovascularization.

82. The method of claim 77, wherein the nanoparticle composition comprises a protease-sensitive, polynucleotide-binding molecule comprising polyethlylene glycol having an average molecular weight of about 2000 daltons, a peptide having the peptide comprising the amino acid sequence Gly-Pro-Leu-Gly-Ile-Ala-Gly-Gln (SEQ ID NO:1), branched polyethylenimine having an average molecular weight of about 1800 daltons, and 1,2-dioleoyl-sn-glycero-3-phosphoethanolamine.

83. The method of claim 77, wherein the nanoparticle comprises a polynucleotide.

84. The method of claim 83, wherein the polynucleotide targets the expression of one or more genes selected from the group consisting of survivin, Eg5, EGFR, XIAP, CDC45L, SUV420h1, WEE1, HDAC2, RBX 1, CDK4, CSN5, FOXM1, R1 (RAM2), LSD1, CSTF2, Nectin-4, ERCC6L, PKIB, NAALADL2, PRMT1, COPZ1, SYNGR4, P-glycoprotein, VEGFR, and VEGF.

85. The method of claim 77, wherein the nanoparticle comprises a hydrophobic pharmaceutical agent.

86. The method of claim 85, wherein the hydrophobic pharmaceutical agent is selected from the group consisting of altretamine, aminoglutethimide, amsacrine (m-AMSA), azacitidine, baccatin III, bleomycin, busulfan, carmustine (BCNU), chlorambucil, cytarabine HCl, dacarbazine, dactinomycin, daunorubicin, docetaxel, doxorubicin, etoposide (VP-16), 5-fluorouracil, floxuridine, flutamide, hydroxyurea, ifosfamide, leuprolide acetate, lomustine (CCNU), melphalan, methotrexate, mitomycin, mitotane (o.p′-DDD), octreotide, paclitaxel, pentostatin, plicamycin, procarbazine HCl, semustine (methyl-CCNU), streptozocin, tamoxifen citrate, teniposide (VM-26), thioguanine, thiotepa, vindesine, vinblastine, and vincristine sulfate.

87. A kit for treating a disease or condition having a cell or tissue that overexpresses a protease, the kit comprising:
(a) the molecule of claim 1; and
(b) packaging therefor.

88. The kit of claim 87, wherein the protease-sensitive, polynucleotide-binding molecule is provided as a dry powder or film.

89. The kit of claim 88, further comprising instructions for reconstituting the protease-sensitive, polynucleotide-binding molecule as micelles in an aqueous suspension.

90. The kit of claim 87, wherein the protease-sensitive, polynucleotide-binding molecule is provided in the form of an aqueous suspension comprising a plurality of nanoparticles comprising the protease-sensitive, polynucleotide-binding molecules.

91. The kit of claim 87, further comprising a polynucleotide.

92. The kit of claim 91, further comprising instructions for forming a nanoparticle composition comprising the protease-sensitive, polynucleotide-binding molecule and the polynucleotide.

93. The kit of claim 87, further comprising a hydrophobic pharmaceutical agent.

94. The kit of claim 93, further comprising instructions for forming a nanoparticle composition comprising the protease-sensitive, polynucleotide-binding molecule and the hydrophobic pharmaceutical agent

95. The kit of claim 87, further comprising instructions for use of the kit.

96. A kit for use in treating a disease or condition having a cell or tissue that overexpresses a protease, the kit comprising:
(a) the nanoparticle composition of claim 23; and
(b) packaging therefor.

97. The kit of claim 96, further comprising a polynucleotide.

98. The kit of claim 97, further comprising instructions for forming non-covalent bonds between the polynucleotide and the nanoparticle composition.

99. A kit for treating a disease or condition having a cell or tissue that overexpresses a protease, the kit comprising the nanoparticle composition of claim 39.

100. The kit of claim 99, further comprising a polynucleotide.

101. The kit of claim 100, further comprising instructions for forming non-covalent bonds between the polynucleotide and the nanoparticle composition.

102. The kit of claim 96, further comprising instructions for use of the kit.

103. A kit for treating a disease or condition having a cell or tissue that overexpresses a protease, the kit comprising the pharmaceutical composition of claim 46.

104. The kit of claim 103, further comprising a polynucleotide.

105. The kit of claim 103, further comprising instructions for forming non-covalent bonds between the polynucleotide and the nanoparticle composition.

106. The kit of claim 103, further comprising instructions for use of the kit.
